(19) United States
(12) Patent Application Publication
(10) Pub. No.: US 2013/0089876 A1
(43) Pub. Date: Apr. 11, 2013

(54) CAPILLARY BIOSENSOR SYSTEM AND ITS METHOD OF USE

(71) Applicants: Omowunmi A. Sadik, Vestal, NY (US); Qiong Wang, Binghamton, NY (US); Paul A. Blythe, SR., Binghamton, NY (US)

(72) Inventors: Omowunmi A. Sadik, Vestal, NY (US); Qiong Wang, Binghamton, NY (US); Paul A. Blythe, SR., Binghamton, NY (US)

(73) Assignee: Research Foundation of State University of New York, Binghamton, NY (US)

(21) Appl. No.: 13/640,896

(22) PCT Filed: Apr. 19, 2011
(86) PCT No.: PCT/US11/33035

§ 371 Date: Dec. 11, 2012

Related U.S. Application Data
(60) Provisional application No. 61/325,663, filed on Apr. 19, 2010.

(51) Int. Cl. G01N 33/566; G01N 33/53

(52) U.S. Cl. CPC G01N 33/566; G01N 33/5302
USPC 435/7.92; 422/69; 436/501; 435/287.2; 435/288.7; 435/29

(57) ABSTRACT

A portable biosensor system includes at least one capillary tube extending longitudinally along a major axis between a proximal inlet end and a distal end. The at least one capillary tube has an interior surface coated with a capture material and forms a waveguide. At least one collimated light emitting diode is disposed proximate and perpendicular to the major axis of the at least one capillary tube and is positioned relative to the at least one capillary tube so that energy enters the at least one capillary tube from its exterior along the entire length of the at least one capillary tube to project a line of energy along substantially the entire longitudinal extent of the at least one capillary tube. A photosensor is disposed proximate the distal end of the at least one capillary tube for receiving emissive radiation therefrom. The photosensor generates an output voltage (or, optical output) representative of the emissive radiation, and a means for measuring the output voltage is provided. Also disclosed is a method of detecting target molecules in a sample using the biosensor of the present invention.

This application claims the benefit of U.S. Provisional Patent Application Ser. No. 61/325,663, filed Apr. 19, 2010, which is hereby incorporated by reference in its entirety.

This invention was made with government support under grant number RD834091010 awarded by United States Environmental Protection Agency (USEPA). The government has certain rights in this invention.

### FIELD OF THE INVENTION

The present invention relates to a capillary biosensor and its method of use.

### BACKGROUND OF THE INVENTION

Biosensors are devices that typically use biological molecules to detect other biological molecules or chemical substances. Detection of food-borne pathogens, biochemical agents, environmental toxins or early cancer biomarkers, requires elaborate time-consuming culturing techniques that must be completed in a microbiology laboratory. Cell-based biosensors (CBBs) can simplify many of these applications and provide better approach towards early detection with improved prognosis. Biosensors have emerged as a promising tool for monitoring cancerous cells or their specific interaction with different analytes. However, the real success in the development of a reliable sensor for cell monitoring depends on the ability to design a powerful instrumentation for efficient signal transduction from cell-to-cell, from cell-to-substrate and from cell-to-extracellular matrices. The resulting sensing system should not affect cell viability, and must function and adapt to the specific conditions imposed by the cascades of cell-based biochemical and biomechanical events, which is a feature missing in conventional biosensor devices and methods.

Specific and/or selective binding interactions with one or more biomolecules (“ligands”) such as peptides, proteins, enzymes, antibodies, receptors, nucleic acids, aptamers, or the like detect one or more target molecules (“analytes”). Binding of the target molecule to the ligand results in a signal that can be used to detect or quantify the analyte present in a sample. The detector molecules are connected in some way to a sensor that can be monitored by a computer or similar mechanism. Biosensors may use a monoclonal antibody to detect an antigen, or a small synthetic DNA molecule called an oligodeoxyribo-nucleotide to detect DNA.

There is a critical demand for a rapid, simple, cost-effective technique for screening samples, such as blood or other clinical samples, for the presence of biomolecules, including polynucleotides, polypeptides, etc. Specifically, the detection of cells, viruses, spores, antibodies, pathogens, or other proteins is considered important in diagnosing and treating diseases. Such detection is also useful for detecting and quantifying such molecules in pathological and forensic samples.

A wide variety of biosensors of different designs is known to those of skill in the art. Such biosensors are designed for use in clinical research laboratories or similar facilities, but tend to be very bulky, expensive, and relatively fragile. Such biosensor systems are typically complex and require highly trained operators to obtain accurate analysis results. Portable biosensor systems based on immunoassays using the optical waveguide as a platform have become an attractive area in sensor research due to the availability of a wide variety of low cost, low power consuming components and bright photostable fluorophores.

In addition to the problems of conventional biosensors discussed above, there have been significant barriers towards the development of cell-based biosensors compared to other sensor components such as antibody, enzymes, and/or nucleic acids. The slow progress could be attributed to limitations resulting from the sensitivity of the cells to environmental fluctuations (such as pH, temperature, and extracellular ion concentrations), sample preparation, stability and limited lifetimes of the cells. Others include maintenance of the biological environment, difficulties with providing reliable fluidic and cell culture media, incompatibility with sensor transducers, inadequate signal generation and processing. Hence, the translation of CBBs from the research labs to real life scenarios at the desired level of sensitivity for practical purposes has not yet been achieved.

Cell viability and motility experiments which traditionally require bulky, controlled environments for cell seeding and survival can benefit enormously from portable incubator with cell culture platform. Several rounds of in-vitro cultures are still required to generate sufficient materials at an appropriate level for conventional drug screening assays. Thawed cultures often fail to grow, while cell populations after passaging may not represent the population within a given sample. Variations in cultured conditions between different laboratories can skew measured EC₅₀ values, thus complicating detection of spatial and temporal trends in cancer prognosis, environmental monitoring or drug screening tests. Current methods of biological automation revolve around the use of liquid handling robots, which are cumbersome and expensive to build and maintain. A portable, capillary-based cell culture platform for in-vitro screening and viability studies should sidestep many of these issues, but has not yet been devised. The present invention is directed to overcoming the deficiencies in the art.

### SUMMARY OF THE INVENTION

According to an embodiment of the present invention, a portable biosensor system includes at least one capillary tube extending longitudinally along a major axis between a proximal inlet end and a distal end. The at least one capillary tube has an interior surface coated with a capture material and forms a waveguide. At least one collimated light emitting diode is disposed proximate and perpendicular to the major axis of the at least one capillary tube and is positioned relative to the at least one capillary tube so that energy enters the at least one capillary tube from its exterior along the entire length of the at least one capillary tube to project a line of energy along substantially the entire longitudinal extent of the at least one capillary tube. A photosensor is disposed proximate the distal end of the at least one capillary tube for receiving emissive radiation therefrom. The photosensor generates an output voltage representative of the emissive radiation, and a means for measuring the output voltage is provided.

A method for detecting presence of a target molecule in a sample includes providing a biosensor system. The biosensor system includes at least one capillary tube extending longitudinally along a major axis between a proximal inlet end and a distal end, having an interior surface coated with a capture material and forming a waveguide, at least one collimated light emitting diode proximate and perpendicular to the major axis of the at least one capillary tube and positioned relative to the at least one capillary tube so that energy enters the at least one capillary tube from its exterior along the entire length of the at least one capillary tube to project a line of energy along substantially the entire longitudinal extent of the at least one capillary tube, and a photosensor proximate the distal end of the at least one capillary tube for receiving emissive radiation therefrom, the photosensor generating an output voltage representative of the emissive radiation. The method includes passing the sample through the at least one capillary tube, directing electromagnetic radiation emitted from the at least one collimated light emitting diode to the at least one capillary tube. The radiation emitted from the at least one capillary tube is received with the photosensor. Any target molecule present in the sample is detected based on the radiation received by the photosensor.

The ultrasensitive portable capillary biosensor of the present invention utilizes the concept of combining optical transduction with a capillary device resulting in low detection limits and better sensitivity compared to planar array biosensors. In this system, capillaries can support various types of immunoassays ranging from simple sandwich types with a fluorescent tagged antibody attached to the surface, to ELISA assays where fluorescent products are enzymatically produced in solution. In binding assays, the capillary serves as a solid support for immobilizing bioreagents as well as an optical waveguide integrating the signal over an increasing surface area. The present invention utilizes wave guiding properties of the capillary to provide signal enhancement to superior detection strategy when compared to conventional ELISA. This technology is an evolution of an optical biosensor from the original bench-top, laboratory-based design to the current portable system.

The system is provided with collimated light emitting diode (CLED). Conventional light emitting diodes are widely used in bio-analytical chemistry for luminescence measurements, providing inexpensive, low-powered and very small sources of radiation, particularly within the wavelength regions where laser diodes are not available. Unlike lasers, LEDs are generally limited by a wide spectral width (15-30 nm), linear output and the light is not tightly collimated so the beam is dispersed over distance and optics. However, this issue is obviated by use of a collimated light source with single or combination of wavelengths the user requires. A user could define a single wavelength such as blue (450-495 nm), green (495-570 nm), or red (620-750 nm) or a combination of these wavelengths depending on the application. Also, the CLED has low power requirements, changeable mode of operation, and performance characteristics.

The capillary arrays provide multiple, individually addressable culture platforms, each with a volume of approximately (18 μL with 7000 cells/capillary). The biosensor system of the present invention also includes inputs for different reagents (e.g. media, staining dyes, buffers, etc) and a peristaltic pump to administer precise doses of these reagents to the capillary chamber. An advantageous feature of the present invention is that seeding a few cells (5-10) per capillary is equivalent to a plating density of 5000-7,000 cells per standard 96 well (0.32 cm²) tissue culture plate. However, the volume of the media per cell in a capillary is ˜60 times lower than in a standard tissue plate culture system. A few μL of suspended cells is more than sufficient to fill the capillary culture chamber, thereby validating the ability to perform cell culture on the capillary from the seeding samples. The user must only insert the sample, turn the pump switch on (or off) to record the data. The rigid alignment of the optical components and the capillary-integrated closed fluidics system can provide a durable setup for field use and can easily be adapted to remote sensing applications. The system of the present invention can also be adapted for multi-analyte detection by the use of patterned capillaries or capillary arrays.

The system of the present invention is a portable, cell culture platform, containing optical detection and control system, a disposable carbon dioxide canister (5% mix), CO₂ monitor with digital display, temperature sensor, a micro heater, and temperature and CO₂ control relays. The detection module of the present invention uses a wavelength selectable, Collimated Dual High Power LED emission sources (filtered, for example, for 504.5 nm and 640.2 nm) with separate driver modules in a single housing known as CLED (collimated light emitting diode). The system of the present invention uses arrays of glass capillaries with precision cell capillary holder. The glass capillaries serve as the template in which mammalian cells can survive, grow, and differentiate. This device provides a powerful tool in which the modulation of cell activities and functions can be systematically controlled. The optical quality of the capillary, wettability, charge, geometrical modification, topographical and spatial control of cells provide positional signals that direct cellular spreading, movement, shapes and ultimately a critical determinant in which cells either survive, become apoptotic or necrotic. The optical and electronic circuitries in the system make it able to achieve broad applications for cell-based biosensors, cell-substrate interactions, cytotoxicity, cytocompatibility, regenerative medicine, tissue culture and engineering.

It is, therefore, an object of the present invention to provide a portable biosensor system that rapidly provides accurate analyses.

It is another object of the present invention to provide a portable biosensor system that is both lightweight and rugged.

It is a further object of the present invention to provide a portable biosensor system that is battery powered.

It is an additional object of the present invention to provide a portable biosensor system that may be used by a relatively unskilled operator to provide accurate measurement.

It is a still further object of the present invention to provide a portable biosensor system that uses a self-contained, digital readout, or a solar panel readout to display analysis results.

It is an additional object of the present invention to provide a portable biosensor system that incorporates an optional interface for exporting analysis results to an external device for analysis or storage.

It is a further object of the present invention to provide a portable biosensor system that uses a capillary treated with a ligand optimized for detection and quantification of a predetermined analyte.

It is yet another object of the present invention to provide a portable biosensor system that uses at least one collimated light emitting diode as an excitation source.

It is a still further object of the present invention to provide a portable biosensor system connectable to a PDA for data collection, wireless monitoring and remote operation.

### BRIEF DESCRIPTION OF THE DRAWINGS

A complete understanding of the present invention may be obtained by reference to the accompanying drawings, when considered in conjunction with the subsequent, detailed description, in which:

FIG. 1A is a schematic system block diagram of exemplary details of the portable biosensor system of the present invention;

FIG. 1B is an internal diagram of a collimated light emitting diode unit used in the portable biosensor system of FIG. 1A;

FIG. 1C illustrates exemplary linear output narrow beam shape, and wavelength colors emitted by the collimated light emitting diode of FIG. 1B;

FIG. 1D illustrates exemplary optical setup details of a biosensor system using the collimated light emitting diode of FIG. 1B;

FIG. 1E illustrates an block diagram of the portable biosensor system according to an alternative embodiment of the present invention;

FIG. 2 is side, elevational, schematic view of the capillary tube, optical arrangement, and photosensor of the biosensor system of FIGS. 1A-1E;

FIG. 3 is a end, cross-sectional view of the capillary tube of the biosensor system of FIGS. 1A-1E;

FIG. 4 is a high level, electrical block diagram of the biosensor system of FIGS. 1A-1E;

FIG. 5 is an electrical schematic diagram of a preferred embodiment of the biosensor system of FIGS. 1A-1E;

FIG. 6 is an exemplary electrical schematic diagram of the system of the present invention;

FIG. 7 shows an example electrical schematic diagram of the LCD readout display unit;

FIG. 8 shows Cyan and Red emission from the system of FIGS. 1A-1E;

FIG. 9 illustrates exemplary CO₂ Concentration Monitor System with Miniature Cylinder and regulator used in the embodiment illustrated in FIG. 1E, according to one embodiment of the present invention;

FIG. 10 illustrates a microprocessor controlled temperature sensor;

FIG. 11 illustrates microphotograph of a PDL coated capillary tube with rat sex cells; and

FIG. 12 illustrates a flowchart for performing an exemplary portable biosensing method.

FIG. 13 is a schematic drawing of the system of the present invention showing further details of the incubator components.

FIG. 14 shows cell viability measurement using Calcein AM (Andreescu, et. al., “High-throughput Biosensor Systems for Monitoring Cells and Bacteria, Encyclopedia of Sensors,” Encyclopedia of Sensors 4: 459-480 (2006) and Srivastava, et. al., Anal. Biochem. 249: 140 (1997), which are hereby incorporated by reference in their entirety.

FIG. 15 shows a readout for a single capillary kinetic study (FIG. 15A) with a standard calibration curve for monitoring cell viability (FIG. 15B). A fixed density of cells which is 2×10⁴ cells per well was seeded to a 96 well tissue culture plate.

FIG. 16 shows the data analysis for monitoring cell viability.

### DETAILED DESCRIPTION OF THE INVENTION

The threat of bioterrorism has spawned a flurry of research focused on developing portable biosensor systems capable of rapidly and sensitively detecting proteins, cells, and other biomarkers. According to an embodiment of the present invention, a portable biosensor system includes at least one capillary tube extending longitudinally along a major axis between a proximal inlet end and a distal end. The capillary has an interior surface coated with a capture material and forms a waveguide. At least one collimated light emitting diode is disposed proximate and perpendicular to the major axis of the at least one capillary tube and is positioned relative to the at least one capillary tube so that energy enters the at least one capillary tube from its exterior along the entire length of the at least one capillary tube to project a line of energy along substantially the entire longitudinal extent of the at least one capillary tube. A photosensor is disposed proximate the distal end of the at least one capillary tube for receiving emissive radiation therefrom. The photosensor generates an output voltage representative of the emissive radiation, and a means for measuring the output voltage is provided.

A method for detecting presence of a target molecule in a sample includes providing a biosensor system. The biosensor system includes at least one capillary tube extending longitudinally along a major axis between a proximal inlet end and a distal end, having an interior surface coated with a capture material and forming a waveguide, at least one collimated light emitting diode proximate and perpendicular to the major axis of the at least one capillary tube and positioned relative to the at least one capillary tube so that energy enters the at least one capillary tube from its exterior along the entire length of the at least one capillary tube to project a line of energy along substantially the entire longitudinal extent of the at least one capillary tube, and a photosensor proximate the distal end of the at least one capillary tube for receiving emissive radiation therefrom, the photosensor generating an output voltage representative of the emissive radiation. The method includes passing the sample through the at least one capillary tube, directing electromagnetic radiation emitted from the at least one collimated light emitting diode to the at least one capillary tube. The radiation emitted from the at least one capillary tube is received with the photosensor. Any target molecule present in the sample is detected based on the radiation received by the photosensor.

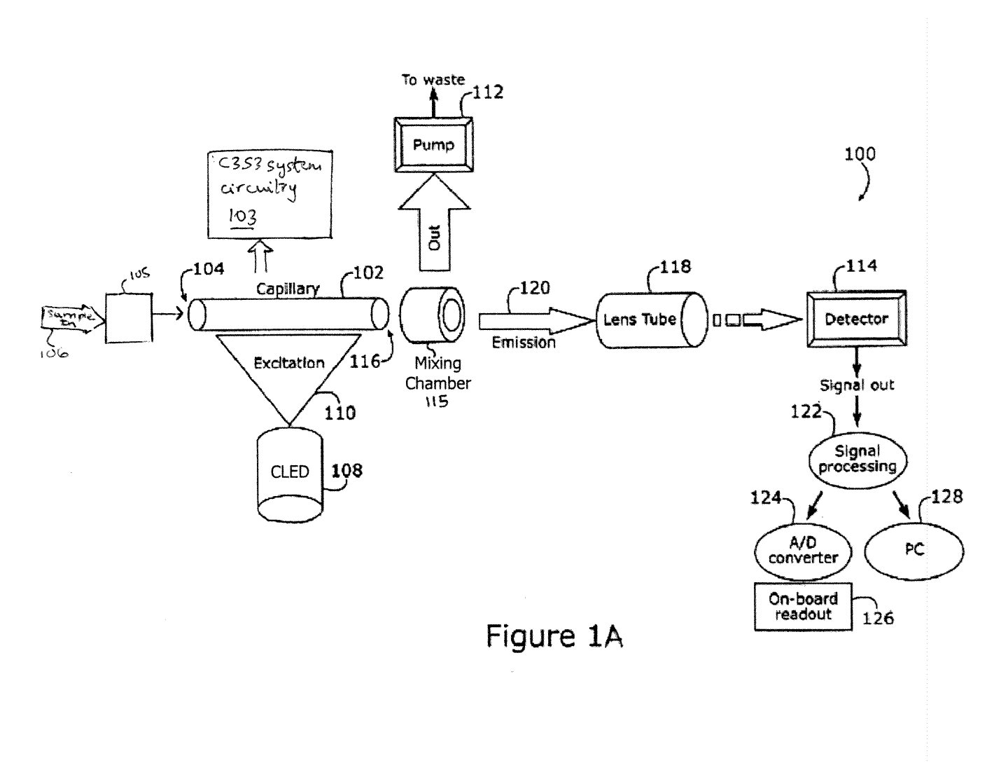

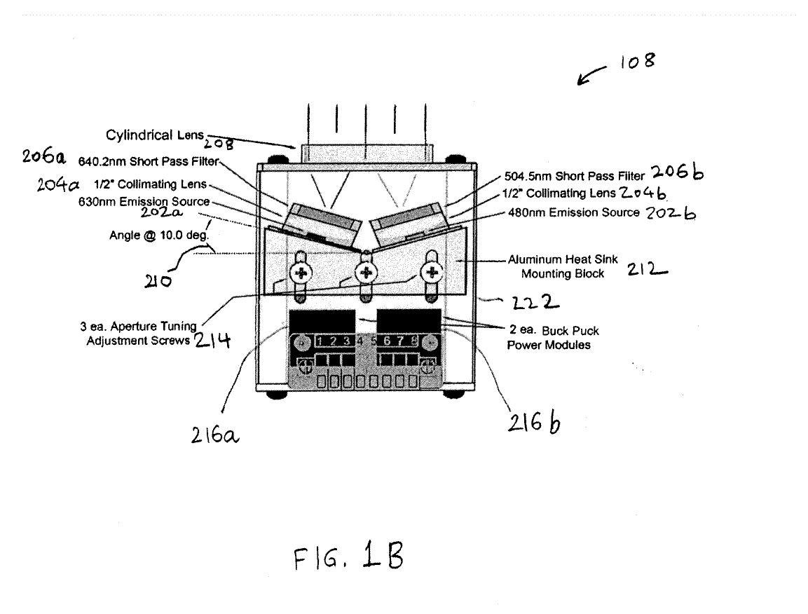

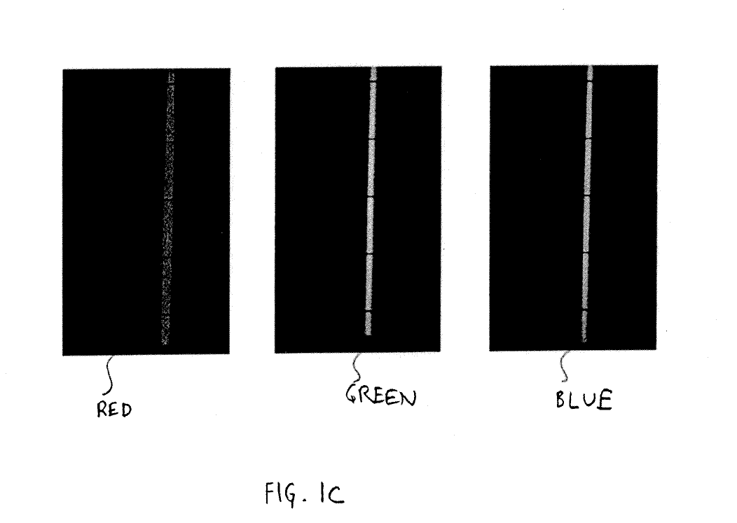

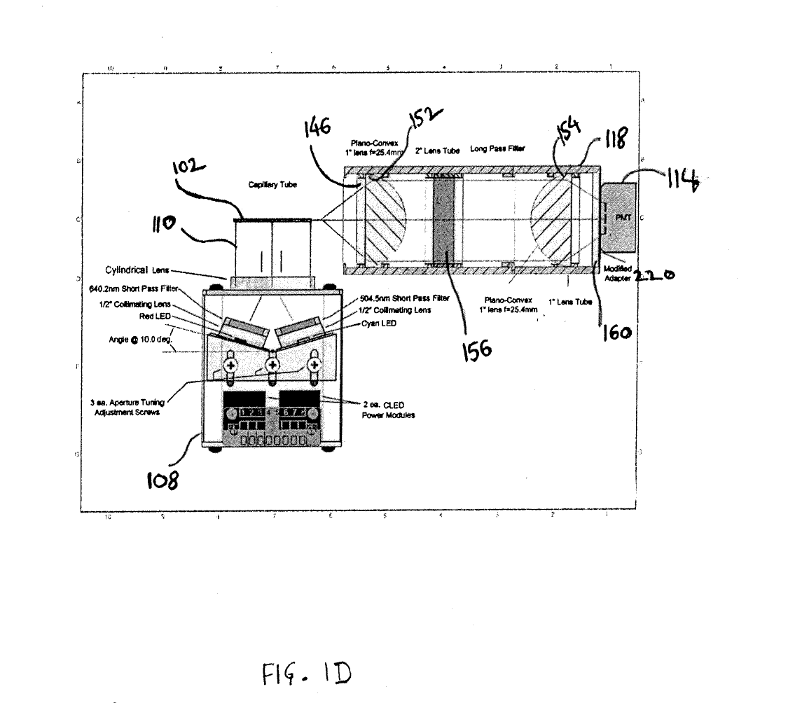

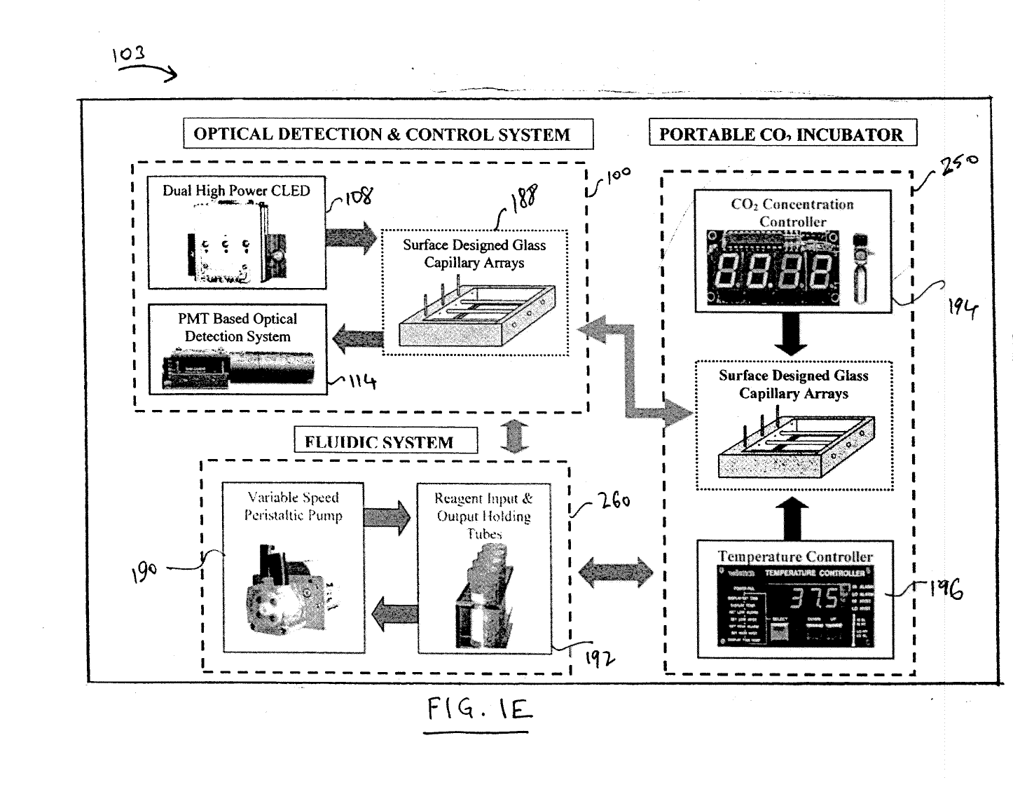

Referring first to FIG. 1A, there is shown a schematic, functional block diagram of the portable biosensor apparatus 100 of the present invention. An at least one capillary tube 102 has a proximal end 104 where a sample 106 to be analyzed may be introduced. Portable biosensor system 100 included on board circuitry for implementing a capillary cell culture screening and survival system (C3S3) 103. At least one capillary tube 102 has an inner surface selectively coated with a suitable immobilized antibody or other suitable capture substance (e.g., RNA, DNA, spores, bacteria, whole cells, aptamers and other ligands). As discussed herein below, the choice of capture antibody is dependent upon the target substance to be detected.

Incubator 105 is positioned to receive sample 106 before it is passed into capillary 104. The incubator serves to grow cells or to increase the amount of materials they produce, which are to be analyzed in the present invention. Operating conditions of incubator 105 correspond to those conventionally used to grow cells.

In use, sterile capillary 104 to be placed in incubator 105 is first coated with positively charged, polycationic polymers such as Chitosan (pKa˜6.5) or poly-D-lysine (PDL). The internal coating with positively charged molecules improves the attachment of adherent type cells, while the capillaries mimic the curvature of the extracellular matrices that most cells experience in their natural environments. Other potential polymeric coatings include poly (ethylene glycol) (PEG), a protein “rejecting” polymer and poly-L-lysine (PLL), which forms a PLL-graft-PEG copolymer that can strongly attach to negatively charged surfaces by coulombic interactions. PEG chains can be further functionalized with integrin—active peptides that contain the Arg-Gly-Asp (RGD) attachment sites. Hydrogen-bonded multilayers comprising of polyacrylamide (PAAm) and weak polyelectrolytes such as poly(acrylic acid) (PAA) can also be used to create patterned, cell resistant/adherent co-culture environments. Polymer coated capillaries are subsequently seeded with mammalian cells to promote cell growth while the cell environments are meticulously maintained at standard conditions of pH and temperature (37° C., 5% CO₂). Finally cell viability is detected with appropriate fluorescent reagents using the available optical components shown in FIG. 1. Viability data are validated against standard approaches such as Tryphan Blue exclusion assays.

Several forms of waveguides in a variety of formats including glass slides (planar waveguides), microarrays, fiber optics, and capillaries have been used as transduction methods for fluorescent immunoassay. Capillaries offer several unique advantages over other waveguide forms or formats. First, it has been shown that the fluorescence signal accumulates along the length of the capillary 102 while the background noise remains substantially constant. This fact provides lower detection limits (i.e., higher sensitivity) compared to other waveguide forms. Second, the capillary 102 is multifunctional. Once the capillary tube 102 is placed in an instrument of system 100, the sensing surface does not come into contact with the outside environment and the capillary becomes an integral part of the flow system. Rinsing and incubation steps may be accomplished by simply pumping the required solution into the instrument. In the case where signal amplification using enzyme-linked immunosorbent assay (ELISA) methods are employed, the capillary also becomes the reaction vessel and the product formation therein can be monitored in real time. Capillary sensors can also be coupled with electrophoresis or patterned for multi-analyte detection. The distal end of the capillary tube holder 102 is a mixing chamber 115 covered, for example, by a replaceable optical grade LEXAN® window provided by Sabic Innovative Plastics of Pittsfield, Mass., although other covers may be used.

A collimated light emitting diode (CLED) 108 in combination with suitable optics, illuminates capillary tube 102 along substantially the entire length thereof with excitation energy shown schematically at reference number 110. For example, a 470 nm or 630 nm wavelength LUXEON® LED provided by Philips Lumileds Lighting Company of San Jose, Calif. has been found suitable for the application. CLED 108 projects a substantially flat, evenly illuminated, 1 mm wide×38 mm long beam, for example, although other dimensions of beam shape may be used depending upon specific applications. CLED 108 is mounted in a pivot mount that also acts as a heat sink. The pivot mount allows the excitation energy 110 to be tightly focused onto the capillary 102 without the need for any intervening, adjustable optical stages. CLED 108 is described in more detail below with reference to FIG. 1B.

In one exemplary embodiment, a line-generating (cylindrical) lens or grating, is placed in front of CLED 108 to spread the circular (collimated) output beam into a line of illumination along capillary tube 102. Such an optical component is well known to those of skill in the optical field and is not further described herein. It will be recognized that a number of suitable optical components exist for performing the beam spreading (e.g., line generating) function; the present invention is not limited to a line-generating lens or grating. Rather, any optical component suitable for forming a line or field of radiant energy 110 along capillary tube 102 may be used.

A pump 112 is provided to both introduce and evacuate the sample to and from capillary tube 102 and, optionally, in cooperation with other apparatus components, to circulate the sample within capillary tube 102. In the embodiment chosen for purposes of disclosure, a miniature, variable-speed peristaltic pump 112, such as Model No. SP100V0, pump manufactured by APT, Litchfield, Ill., was utilized. The pump 112 is connected to a 3-way switch, or other suitable control that permits selective operation of pump 112 at flow rates of approximately 0.18 ml/min (high speed) and 0.4 ml/min (low speed). The faster flow rate has been found useful for performing rinsing steps as described hereinbelow. It will be recognized that other suitable pumps or pumps having different flow rates may be known to those skilled in the art and may be substituted for the APT pump used for purposes of disclosure. The present invention is not considered limited to any particular pump or specific flow rates. Rather, the present invention covers any and all suitable pumps and/or flow rates.

A photosensor 114 is disposed proximate a distal end 116 of capillary tube 102 via optical arrangement 118. Photosensor 114 monitors the fluorescence of the excited sample 106 within capillary tube 102, shown schematically as emission 120, and generates an electrical signal representative thereof. A photomultiplier tube or other sensitive photosensor may be used. Examples of other photosensors include photodiodes and infrared detectors, for example, Michelson Interferometers.

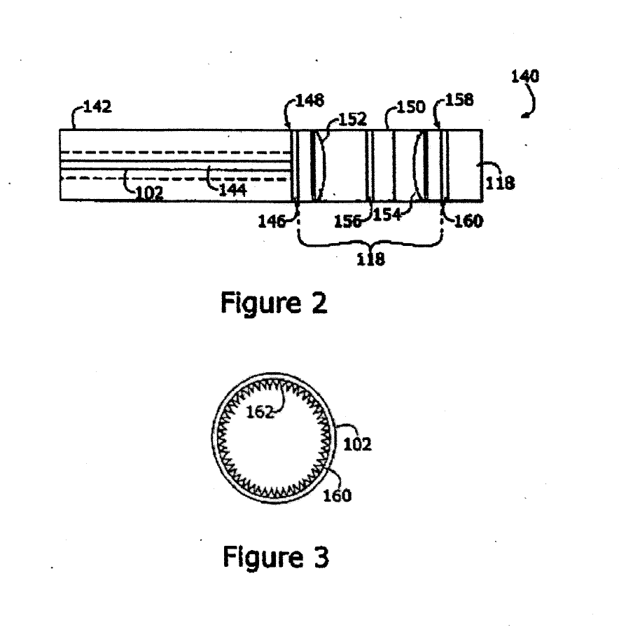

In the preferred embodiment, optical arrangement 118 and detector (i.e., photosensor) 114 are axially aligned with the central, longitudinal axis of capillary tube 102. In the embodiment chosen for purposes of disclosure, a conventional lens tube 150 (FIG. 2) known to those of skill in the optical arts is used to support and align a pair of plano-convex lenses 152, 154 (FIG. 2) to focus and concentrate emission light 120 at a receiving surface of photo detector 114. It will be recognized that other optical arrangements, likewise, may be used. It will further be recognized that a fiber optical coupling could be inserted between distal end 116 of capillary tube 102 and other optical arrangement 118 or photosensor 114. Consequently, the present invention is not limited to the particular photosensor 114, optical arrangement 118, or placement of the photosensor relative to capillary tube 102 chosen for purposes of disclosure. Rather, the present invention covers any and all suitable photosensors 114, optical arrangements 118, and relative positions of photosensors 114 to capillary tube 102.

Signal processing electronics 122 is operatively connected to photosensor 114 and receives an electrical signal therefrom. An analog-to-digital (A/D) converter and associated circuitry 124 is used to drive an on-board display or readout 126 where quantitative/qualitative information regarding a sample being tested is displayed. In alternate embodiments, an optional interface 128 may be provided to allow attachment of a computer (e.g., a notebook computer, PDA, etc.) or other external device useful for processing, correlating, post analyzing, or otherwise processing and/or storing result data. Such interfacing may be accomplished in a wide variety of ways including, but not limited to, serial and parallel direct connections, infrared communications ports, network (including wireless) connections, proprietary interfaces, and the like. As these interfacing techniques are considered well known to those of skill in the computer arts, they are not further described herein. The present invention is seen to encompass any viable communication strategy.

In operation, reagents, are introduced into capillary tube 102 adjacent proximal end 104 where they interact with the immobilized antibody 162 (FIG. 3) or other suitable capture substance coated upon the inner surface 160 thereof. Once the target analyte is captured, a fluorescent labeled antibody, a fluorescent labeled avidin, or a fluorescent ELISA using an avidin/alkaline phosphatase complex is introduced into capillary tube 102. For fluorescence detection, Alexa-Fluor 647 is preferred due to its improved performance over Cy-5 when used to label tracer antibodies for sandwich immunoassays. As mentioned hereinabove, other materials may be substituted for Alexa-Fluor 647. Also any other similar, suitable substances known to those of skill in the art may be substituted therefor.

Referring now to FIG. 1B, exemplary details of CLED 108 are described. CLED 108 is arranged internally to such that emitters or electromagnetic emission sources 202a and 202b have a relative angular orientation with respect to each other resulting in a unique geometry of the CLED 108. CLED 108 provides a laser-like performance that was not previously available with conventional LEDs, especially in the lower wavelengths.

In this example, CLED 108 is a high intensity illumination source including two wavelength switch selectable Collimated Dual High Power LED emission sources 202a and 202b (operating substantially at 490 nm and 635 nm) with separate driver modules 216a and 216b, respectively, in a single housing 222. Each emitter source 202a, 202b is equipped with a Short Pass (Edge) Filter 206a and 206b, respectively, collimating lenses 204a and 204b, respectively, and a cylindrical lens 208 that is optimized for line illumination of 38 mm at 36 mm distance. The line illumination length, width, and power, can be mechanically adjusted by both the aperture-tuning adjustment screws 214, and the CLED mounting bracket set screws. The purpose of the Short Pass Filters 206a, 206b is to allow only the desired excitation wavelength to pass, as illustrated in FIG. 1C, for example. In another example, FIG. 1C illustrates the “Red”, “Green”, and “Blue” linewidths output from CLED 108, although other colors or wavelengths may be used. Alternatively, in other examples, the CLED 108 may be designed to accommodate additional emission sources of varying wavelengths and intensities, as can be contemplated by one of ordinary skill in the art, in view of this disclosure.

Although the typical LED Spectral Half-width is between 20-30 nm, the new high powered (50 mw or higher) type CLED 108 maintains a tolerance of +/−0.5 nm for dominant wavelength measurements. To maintain a fine granularity with the overall flux distribution, the lower wavelength (Royal-Blue) LED's are binned by radiometric power and peak wavelength rather than photometric lumens. Another advantage of the high power CLED 108 is when using the new high power modules, the output is no longer linear with the supply current. High power LEDs (e.g., CLED 108) maintain a tolerance of +/−2 nm for peak wavelength measurements, and have a total angle at which 90% of total luminous flux is captured.

CLED 108 can be powered by a wide range LED power modules, also referred to as LED drivers. For example, the power modules 216a and 216b selected in this example were each LUXDRIVE™ 3021 Buck Puck provided by LEDdynamics, Inc. of Randolph, Vt. These are a line of true current regulated LED drivers. These compact LED drivers 216a and 216b are high efficiency dc to dc converters which deliver a fixed output current by varying the output voltage. This arrangement provides a fast response current-sensing circuitry with a wide range of voltage and intensity controls. When used in conjunction with an external adjustment potentiometer, it provides a 0%-100% control of output intensity (when Vᵢₙ>5.25 V_{DC}). Each power module 216a and 216b can drive multiple high power LEDs (e.g., CLED 108) depending on input voltage. In one example, two different modules 216a and 216b can be used to account for different voltage and intensity curves (for CLED and PMT Trimming) of the two wavelengths selected.

Referring to FIG. 1D, an overall optical design concept of the system of the present invention, according to an embodiment of this invention is disclosed. The overall optical design concept consists of four example components: the CLED 108 including emission sources 204a and 204b, capillary tube holder 102, main lens tube 118, and photo-multiplier tube (PMT) 114. Depending on the choice of emission source selected (480 nm or 630 nm), the light is collimated and filtered by short pass filters 206a and 206b with cut-off wavelengths (CWLs) of either 504.4 nm (Cyan) or 640.2 nm (Red), then passed through a cylindrical (line generating) lens 208 to result in line patterns discussed above with reference to FIG. 1C, and focused onto a bottom surface of the capillary tube 102. The distal end of the capillary tube holder 102 is a mixing chamber 115 covered, for example, by a replaceable optical grade LEXAN® window provided by Sabic Innovative Plastics of Pittsfield, Mass., although other covers may be used. A two piece lens system forming lens tube 118 is focused on the optical center of the capillary tube holder 102 window is used to detect the fluorescence signal from the capillary tube 102. The output signal from this lens tube 118 is coupled to a Photomultiplier Tube (PMT) 114 by means of a specially designed adapter 220 arranged to maintain optical centering and rigidity.

A two piece lens system forming lens tube 118 includes a 2″ long front lens tube and a 1″ long rear lens tube, and is used to facilitate the changing of the Long Pass filter 156. The rear 1″ lens tube remains attached to the PMT 114, while the 2″front lens tube (with Long Pass filter 156) is replaced when selecting a different wavelength light (excitation) source. For each light (excitation) source, there is a Long Pass filter 156 for the corresponding emission wavelength (e.g., Red is 660 nm and Cyan is 510 nm). The Long Pass filters 156 are selected to allow only wavelengths at, and above the wavelength of the excitation signal to pass to the PMT 114. For example, one commercially available cell viability staining kit contains Calcein AM, a widely used a widely used green fluorescent cell marker. Calcein AM excitation wavelength is 488 nm, and its emission wavelength is 515 nm. In the system 100 of the present invention, a 504.5 nm short pass filter is used in the CLED 108 light (emission) source, and a 510 nm long pass filter located in the 2″ front lens tube. Further details of the two-piece lens tube 118 are discussed below with reference to FIG. 2.

Referring to FIG. 1E, in an alternative exemplary embodiment of the present invention as included within C3S3 system 103, system of the present invention 100 is illustrated coupled to a portable CO₂ incubator system 250 and a fluidic system 190. System of the present invention 100 is substantially similar to the system shown in FIG. 1A, except as described herein below. The system of the present invention 100 in this example embodiment includes a plurality of capillary tubes in a surface designed glass capillary array 188 instead of a single capillary tube 102 shown in FIG. 1A. An example advantage of using the array 188 is that a plurality of capillary tubes can be processed in parallel leading to a faster cell culture analysis time. Array 188 may include addressing means implemented by computer control, moving and storage means, as will be apparent to one of ordinary skill in the art after reading this disclosure. Collimated wavelengths of light from CLED 108 in the form of excitation 110 are projected on the array 188 and the output electromagnetic radiation, e.g., emission 120, emitted by array 188 in response to the excitation 110 are detected by PMT based optical system including PMT 114, optically coupled to the output of the array 188, although other forms of coupling may be used.

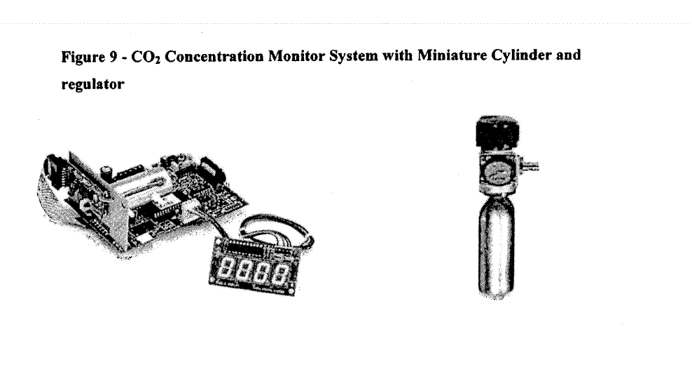

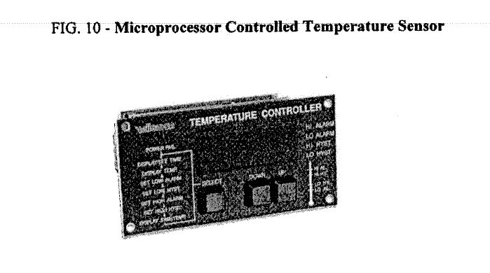

The system of the present invention 100 is coupled to the fluidic system 260 including a variable speed peristaltic pump 190, although other types of pumps, for example, pump 112 of FIG. 1A, may be used. Pump 190 communicates fluidically and electro-mechanically in a bi-directional manner to provide input to array 188's capillaries and output to output holding tubes in a reagent input and output holding tube module 192. Reagent input and output holding tube module 192 are maintained at a stable temperature using, by way of example only, a temperature controller 196, although additionally temperature controller 196 may be used to maintain a temperature of array 188. Further details of temperature controller 196 are provided with respect to FIG. 10 below and are not being repeated here. The system of the present invention 100 is optionally coupled to the portable CO₂ incubator system 250 for maintaining controllable CO₂ levels using a CO₂ concentration controller 194. Details of portable CO₂ incubator system 250 are provided with respect to FIG. 9 below and are not being repeated here.

Referring now to FIG. 2, there is shown a side, elevational, schematic view of capillary tube 102 in a support structure, generally at reference number 140. In the embodiment chosen for purposes of disclosure, capillary tube 102 is a fused silica capillary approximately 38 mm long having an inside diameter of approximately 0.78 mm and an outside diameter of approximately 1.0 mm, by way of example only. The capillary tube 102 is available from Polymicro Technologies, Phoenix, Ariz.

Capillary tube 102 is mounted in a custom scaffold or precision capillary holder 142 that contains a longitudinal window that allows the line excitation 110 to illuminate capillary 102 along substantially the entire length thereof. Emission (e.g., electromagnetic radiation) is collected through an optical grade LEXAN® window 146 disposed at a distal end 148 of precision capillary holder 142. In the embodiment chosen for purposes of disclosure, precision capillary holder 142 is formed from LEXAN®. LEXAN® was chosen for its weight, rigidity, and ease of machining in constructing prototypes. However, it will be recognized that other lightweight but rigid material such as computer-milled aluminum and Teflon with 70% glass may also be easily substituted for LEXAN®.

A stackable lens tube 150, obtained from Thorlabs, Newton, N.J., is abutted to and axially aligned with the transparent window 146. In the embodiment chosen for purposes of disclosure, lens tube 150 is approximately 3 inches long and has a diameter of approximately 1 inch. Lens tube 150 comprises a 2″ front tube and a 1″ rear tube and supports optical components, for example a pair of plano convex lenses 152, 154 and a long-pass interference filter (e.g., a 650 nm low-pass filter 156 obtained from Omega Optical, Brattleboro, Vt.). The 2″ front lens tube 150 includes a plano-convex lens and a long pass filter, for example. The lens tube 150 is changed when CLED 108 wavelength changes because it contains the wavelength specific long pass filter. The rear part of lens tube 150 (1″ in length) is fixed to photosensor 114. Lenses 152, 154 and filter 156 form optical arrangement 118 as shown in FIG. 1A. The lens tube 150 is threaded on the inside. Retaining rings are used to hold the optics 152, 154, 156 in place therein. This arrangement allows the optical components 152, 154, 156 to be optimally distance-adjusted with respect to one another, to the end of capillary tube 102, and to photosensor 114, respectively, and then secured in place within lens tube 150.

Photosensor module (i.e., photodetector) 114 is attached to distal end 158 of lens tube 150 via a custom made aluminum mounting adapter 160. A Catalog No. HC-5784-20 photosensor manufactured by Hamamatsu (Japan) has been found suitable for the application. Aluminum mounting adapter 160 includes external threads that screw into the distal end of the 1″ length portion of lens tube 150, and four mounting screw holes that attach it directly to photosensor 114.

The photosensor module 114 contains a photomultiplier tube, a built-in high voltage power supply, and a low noise amplifier that converts the output current from the photomultiplier tube to voltage representative thereof. When compared to a well-known Hamamatsu HC 120 bench top analyzer, it was found that the 5784 photosensor 114 exhibited lower noise than did the HC 120 instrument. However, 5784 photosensor 114 had lower gain resulting in decreased detector sensitivity. The lower sensitivity can be overcome substantially by improving the optical mounting components, internal lens focus distance, and using a higher power (e.g., 15 mW (or greater) vs. 12 mW) CLED 108 in the portable instrument.

The rigid alignment provided by the sensor platform (i.e., capillary tube 102/precision capillary holder 142) optical arrangement 118, and photodetector 114 typically were found to require no further alignment adjustment after assembly. The arrangement has been found adequate for field use where the system may be exposed to some shock during transportation and use.

Referring now to FIG. 3, there is shown an end, sectional, schematic view of capillary tube 102. The inside surface 160 of capillary tube 102 is coated with capture material (e.g., an immobilized antibody, etc.) 162 as described in detail hereinbelow. Many different materials may be immobilized on interior surface 160 of capillary tube 102. The selection of capture material 162 depends, of course, upon the analyte to be detected and/or quantified. For example, goat anti-mouse IgG, mouse IgG whole molecule, biotinylated goat anti-mouse and the phosphatase substrate para-nitrophenyl phosphate (PNPP) may be utilized as required. The aforementioned materials are available from Pierce Biotech, Rockland, Ill.

The target analyte is captured by immobilized antibody 162 on the inner surface 160 of capillary tube 102 and then detected using a fluorescent labeled antibody, a fluorescent labeled avidin, or a fluorescent ELISA in conjunction with an avidin/alkaline phosphatase complex. For fluorescence detection, Alexa-Fluor 647 was selected due to its reported improved performance over Cy-5 when used to label tracer antibodies for sandwich immunoassays. It will be recognized, however, that a single capture antibody 162 may be used. In alternate embodiments, the system can also be adapted for multi-analyte detection by the use of a patterned capillary tube 102 wherein more than one capture antibody 162 may be applied to surface 160. In still other embodiments, multiple capillary tubes 102, each coated with a different capture antibody 162 and disposed in a parallel arrangement, may be used to detect multiple analytes in a sample.

For purposes of disclosure, three different immunoassay formats using fluorescent labeled proteins, avidin/biotin chemistry, and enzyme linked immunosorbent assays are described. These methods were optimized using mouse IgG as the target antigen. The results of each assay were compared with each other as well as with the results of a conventional colorimetric ELISA assay performed in a 96-well plate.

Capillary tubes 102 were first prepared assuming the desired analyte to be goat anti-mouse IgG by serially interconnecting multiple capillary tubes 102 using Tygon™ tubing. Solutions were drawn into the string of capillary tubes 102 using a plastic syringe A syringe having a toluene-resistant plastic plunger and a lure-lock tip were found suitable.

Immobilization of the goat anti-mouse capture antibody was achieved using covalent chemistry well known to those of skill in the art. After a sequence of cleaning steps using approximately 50-50 ratio of methanol/HCl and sulfuric acid, the capillary tubes 102 were incubated under nitrogen with a 2% solution of 3-mercaptopropyl trimethoxy silane in anhydrous toluene. The capillary tubes 102 were then treated with the hetero-bi-functional cross linker (N-[ã-maleimidobutyryloxylsuccinimide ester)(GMBS). Goat anti-mouse capture antibodies at a concentration of 10 μg/ml in phosphate buffered saline (PBS) were then attached to the capillary tube 102 via the crosslinker by an overnight, refrigerated incubation.

For direct assays, a 10 μg/ml solution of goat IgG whole molecule was immobilized in place of the anti-goat capture antibody. Before use, the capillary tubes 102 were blocked with a 1 mg/ml solution of BSA. For a direct comparison, conventional colorimetric sandwich ELISA assays were performed using the same antibody-antigen combination as used in the capillary tubes 102. A 96-well micro-titer mouse in pH 9.6 carbonate buffer treated for 2 hours at room temperature was performed. The micro-titer plates were rinsed thrice and blocked with a 10 mg/ml BSA solution in PBS with another 2-hour incubation at room temperature.

After preparation with the capture antibody as described hereinabove, the ELISA plates were exposed to the antigen (mouse IgG) standards for 1 hour followed by a rinse step and an hour exposure to 10 μg/ml solution of biotinylated goat anti-mouse. After another rinse step, the wells were exposed to the avidin/alkaline phosphatase substrate in pH 8.0 Tris buffered saline with 5 mg/ml BSA for 1 hour. The enzyme complex was titered at ratios of 1:5000, 1:10,000, 1:20,000 and 1:40,000. The PNPP substrate in DEA buffer, pH 9.6, was added to each well and incubated for 20 minutes. Plates were then read at 405 nm on a Biotek Elx800 microplate reader.

Direct binding assays were initially performed using the portable instrument of the present invention to compare the signal generated by an avidin-Alexa Fluor 647 conjugate and an avidin-alkaline phosphatase complex coupled with DDAO-phosphate as a substrate. In this scheme, the mouse IgG was immobilized at a constant concentration in the capillary tube 102 and the goat anti-mouse/biotin (GAMB) was diluted and used as the standard to be detected. For direct ELISA assays, the GAMB standards (prepared in phosphate buffered saline with 1 mg/ml BSA and Tween 20 (PBSTB)) was drawn into the capillary 102 using a plastic syringe and then incubated for approximately 15 minutes.

The capillary 102 was then rinsed with PBSTB and a 1:20,000 solution of the avidin/AP complex in pH 8.0 tris buffered saline with 5.0 mg/ml BSA was added and incubated for approximately 5 minutes. The capillary 102 was then inserted into the instrument and a buffer was flowed therethrough through at a flow rate of approximately 0.19 ml/min. After a few seconds, the inlet was switched over to the substrate (20 μM DDAO in pH 9.8 Tris buffer with 100 mg/L MgCl₂). As used herein, the term substrate refers to the molecules used for amplification of immunological reactions commonly used in Enzyme linked Immunosorbent Assay (ELISA). ELISA is considered the “gold standard” for immunological analytical techniques. In an ELISA, an antibody (primary) specific to an antigen (or target species) is immobilized onto a solid support such as a polystyrene plate microwell plate. The antigen, (or target species) specifically binds to the capture antibody. A labeled second antibody (secondary) specifically recognizes another epitope on the antigen (or a site on the target). The secondary antibody is conjugated to an enzyme and doubles up as the detection antibody. The final step of the assay is amplification, which is made possible by the addition of a substrate upon which the enzyme acts with a very high turnover rate giving a detectable product. The endpoint of the enzymatic reaction, typically leads to a colored product that is detected spectrophotometrically. The absorbance is used to quantify the amount of antigen or target species, present in the sample.

When the substrate passed into the capillary, the pump 112 was shut off and the enzymatic cleavage of DDAO was allowed to proceed.

For direct assays involving avidin-AF647, the GAMB standards were incubated in the same manner as described hereinabove. However, after the incubation step, the capillary 102 was placed in the potable instrument. Buffer was flowed through the capillary 102 and a baseline voltage was recorded for approximately 20 seconds. A solution of avidin-AF647 (10 μg/ml in PBSTB) was then introduced into capillary tube 102. After the avidin-AF647 solution had entered the capillary tube 102, the pump 112 was switched off and incubation of approximately 5 minutes was allowed. After the incubation period, buffer was reintroduced into capillary tube 102 and the pump 112 was operated at high speed for approximately 30 seconds. Following the buffer rinse, the electrical output signal was recorded with an increase in voltage being indicative of surface bound AF647.

Capillary sandwich fluorescent ELISA assays were performed using the capillaries 102 with immobilized goat anti-mouse IgG. Capillaries 102 were strung onto syringes using Tygon tubing. An incubation sequence having the indicated steps was then performed:
i) incubate with mouse IgG standard;
ii) incubate with GAMB secondary antibody (10 μg/ml); and
iii) incubate with avidin-AP complex.

Appropriate rinses were performed between each of the incubation steps by drawing a buffer solution into the syringe, disconnecting the capillaries 102, and discharging the buffer solution. The sequence was repeated two additional times. After the final incubation, the capillary 102 was inserted into the instrument and the DDAO substrate was flowed in as in the direct assay method described hereinabove. For optimization, different incubation times ranging between approximately 5 and 60 minutes, and different titers of the enzyme complex (1:5,000 to 1:30,000) were used. These parameters, in conjunction with controls containing no capture antibody, no antigen or no biotinylated secondary antibody were used to determine an optimum assay. Assays using the avidin-AF647 complex were executed using the following sequence:
i) incubate with mouse IgG standard for 10 minutes;
ii) incubate with GAMB secondary antibody for 5 minutes (10 μg/ml);
iii) insert capillary 102 into instrument and record a baseline;
iv) flow in avidin-AF647 complex and incubate for 5 minutes; and
v) rinse for 30 seconds and record signal while flowing buffer at 0.18 ml/min.

Controls used were identical to those described for the fluorescent ELISA hereinabove.

Sandwich assays using the AF-647 labeled goat anti-mouse tracer antibody were performed in a similar fashion. After incubation with the antigen standards for 10 minutes, the capillary 102 was inserted into the instrument. A solution of 10 μg/ml AF-647 labeled goat anti-mouse in PBSTB was introduced into capillary tube 102. The pump 112 was shut off and the antibody was allowed to incubate for various times in the range of between approximately 4 and 15 minutes to optimize the signal to noise ratio. Controls consisted of capillary tubes 102 prepared with no capture antibody as well as blank capillary tubes 102 with no antigen present.

Materials for use in evaluating and/or operating the biosensor system of the present invention are available from several sources. Alexa-Fluor 647 NHS-ester, Alexa Fluor 647 labeled streptavidin and the phosphatase substrate 9H-(1,3-dichloro-9,9-dimethylacridin-2-one-7-yl)phosphate, diammonium salt DDAO-phosphate) were purchased from Molecular Probes, Eugene, Oreg. Goat anti-mouse antibodies to be used as tracers were labeled with a 15-fold molar excess of Alexa-Fluor 647 NHS-ester at pH 8.0 and incubated overnight in the refrigerator. Unbound AF 647 was removed with protein desalting spin columns (Pierce Biotech, Rockland, Ill.) according to the manufacturer's instructions. The dye:protein ratio was determined to be 4.2:1 by measuring the absorbance at 280 and 650 nm on a Hewlett-Packard diode array spectrophotometer and making the calculations according to the manufacturer's instructions. Bovine Serum Albumin (BSA) and alkaline phosphatase labeled avidin were purchased from Sigma, ST. Louis, Mo.

The system of FIGS. 1A-1E can be miniaturized for portable applications. It is desirable that such a portable instrument exhibit four important characteristics. First, size and weight should be minimized to create in instrument readily usable in the field. Ideally, all power for the instrument should be supplied by internal batteries, preferably rechargeable batteries. Second, the instrument must be rugged to withstand rough handling to which such an instrument is typically subjected. As the instrument may contain a fragile capillary tube and optical components requiring relatively precise alignment, proper shock mounting of components is required. Third, the instrument should be sensitive to allow precise quantitative/qualitative measurements to be performed in as short an amount of time as possible. Finally, the instrument should be relatively inexpensive.

The prototype used for purposes of disclosure fulfills these four requirements. The prototype exhibits a weight of approximately 33.5 pounds, or 15.4 kg, and is packaged in approximately a 12×4×5 inch volume. It is believed that the size of the instrument may be further reduced, ultimately to the size of a typical PDA or similar hand-held instrument.

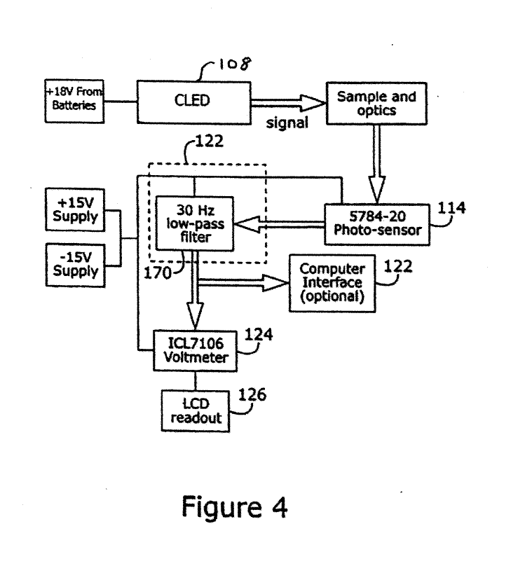

Referring now to FIG. 4, there is shown a system block diagram of the portable biosensor of the present invention. The electronic signal processing portion 122 includes an low-pass filter 170, an integrating voltmeter 124, a self-contained digital readout 126, and an optional computer interface 122.

An electrical signal output of photosensor 114 is connected to the input of low-pass filter 170. In the embodiment chosen for purposes of disclosure, low-pass filter 170 is implemented as a Butterworth filter including an operational amplifier (op-amp) having an appropriate feedback network to form the desired cut-off frequency and slope, although other types of filters may be used (e.g., Chebysheff filters). Butterworth filters are well known to those of skill in the electronic design arts and are not further described herein.

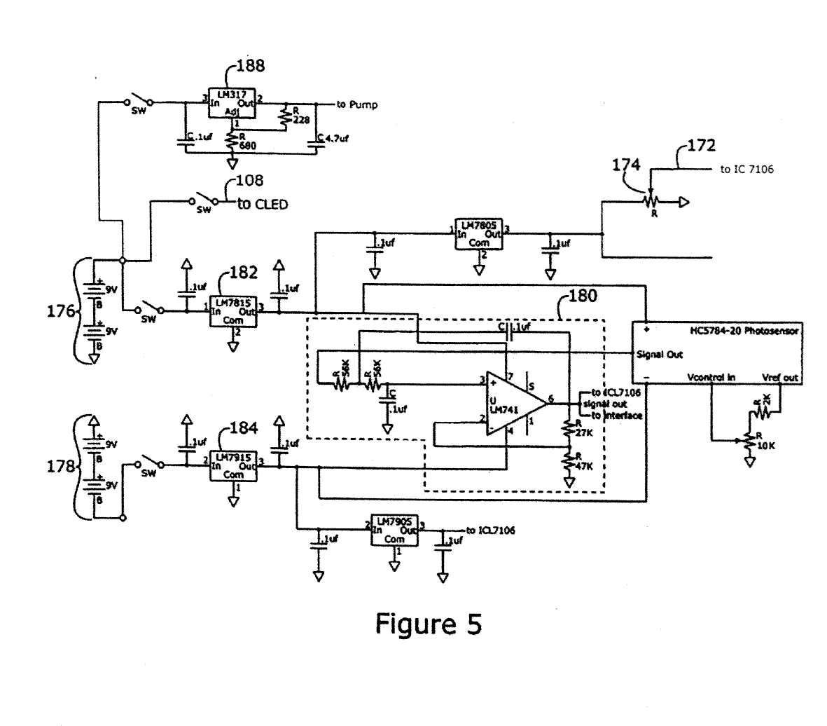

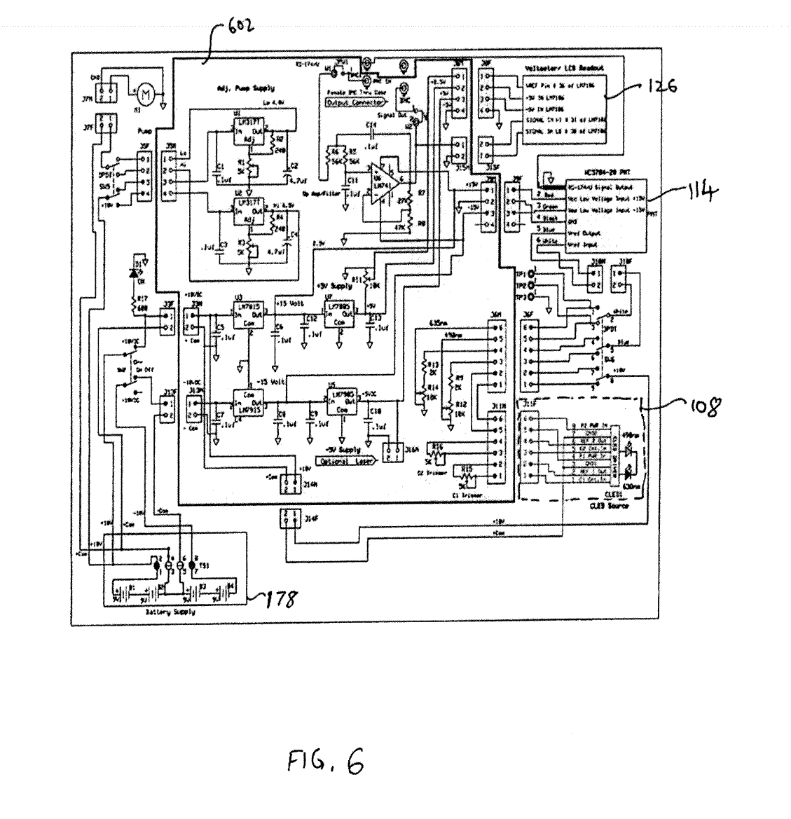

Referring now also to FIG. 5, there is shown an exemplary circuit diagram of an embodiment of the inventive, portable biosensor system. In the illustrative prototype, an LM-741 op-amp is used. For purposes of disclosure, a filter circuit having an approximately 30 Hz cut-off frequency has been chosen. It will be recognized that other suitable low-pass filter topologies and or implementations may be substituted for the Butterworth filter chosen for purposes of disclosure. Consequently, the present invention is not considered limited to any particular filter design. It will be further recognized that circuit designs may be provided without any low-pass filter. The present invention is intended to include such designs as well.

The output of low-pass filter 170 is connected to the input of an A/D converter forming an integrating voltmeter 124. The output of integrating voltmeter 124 is connected to the input of a digital display device 126 (i.e., a digital readout). Integrating voltmeter 124 may be implemented using an IC7106 analog-to-digital (A/D) converter adapted to directly drive an LCD display device 126. The IC7106 chip accepts an absolute voltage reference (available from the power supply of the portable instrument) using a calibration potentiometer 174 or other suitable arrangement. This allows accurate, absolute voltage measurements to be performed, typically at a rate of approximately 3 readings per second. Reading capability in the range of 0-5 volts has been found satisfactory, even when high concentrations of fluorescent dyes are utilized for an assay. The digital readout 126 of the apparatus has been found to be suitably stable to allow manual recording of readings by an operator of the instrument. A 3.5-digit digital display has been found to be adequate.

The prototype can be operated with a power supply of both plus and minus voltages in the range of approximately 12-15V 176, 178, respectively. Voltage regulators 182, 184 maintain a constant voltage to the circuitry as output voltage from batteries 176, 178 decreases. Series-connected 9-volt batteries have been found suitable to provide voltages 176, 178.

To reduce current draw, the +18VDC is taken directly from the batteries to power the CLED 108. Battery lifetime is typically not a major concern as both pump 112 and CLED 108 are intermittently operated. Although it is possible to power CLED 108 from power supply 176, it is desirable to use the battery voltage because there are no transients and the CLED will draw less current at 18VDC then +15VDC of the on board regulator.

The pump is controlled by two identical voltage regulator circuits 188, one for high speed and the other for low speed. The output signal from the low-pass filter 170 is fairly clean and typically does not require complex lock-in amplification or other specialized signal processing. The photosensor module or PMT 114, CMOS A/D converter (i.e., integrating voltmeter) 124, filter circuit 180 and LCD display 126 are all driven by the same power supply, typically consisting of four 9-volt batteries. In theory, the power supply can last for a maximum of 70 hours while powering all of the above components. However, if pump 112 and CLED 108 (FIGS. 1A-1E) are run from the same power supply 176, typical battery lifetimes are reduced to approximately 10 hours. As previously stated, either non-rechargeable or rechargeable batteries may be used. Rechargeable batteries are preferable and built-in recharging capability, may be provided if desired. The low power consuming photosensor module 114 makes possible a biosensor containing two or more of these photosensor modules 114 configured for multi-analyte or multiple sample analysis feasible.

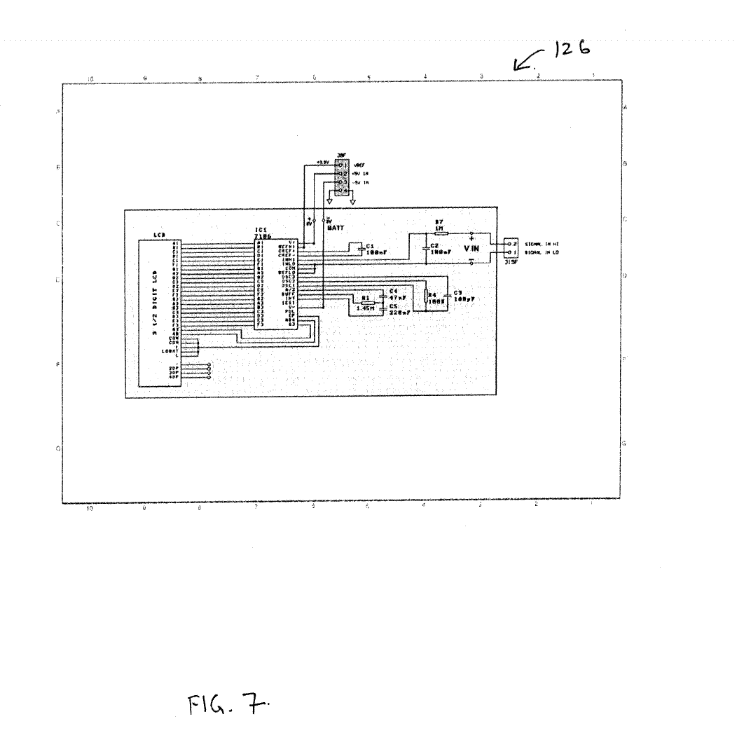

Referring to FIG. 6, an alternative electrical schematic in accordance with some other embodiments of the present invention its interconnecting components is illustrated. Area 602 is on the PCB, and the peripheral systems are all attached by connectors to allow for easy removal. This schematic shows only the battery power source 178 consisting of 4 each 9 V_{DC} batteries. An AC adapter power source is also available as are rechargeable batteries. As illustrated in FIG. 6, interconnections between an LCD readout 126, CLED 108, and photomultiplier PMT 114 and various driving, regulating, and amplifying circuitry on area 602 is illustrated.

Referring to FIG. 7, an exemplary electrical diagram schematic for an LCD readout to display various readings of the system 100 of the present invention is shown. The LCD readout illustrates standard electronic components known to those of ordinary skill in the art, and will not be described in detail herein.

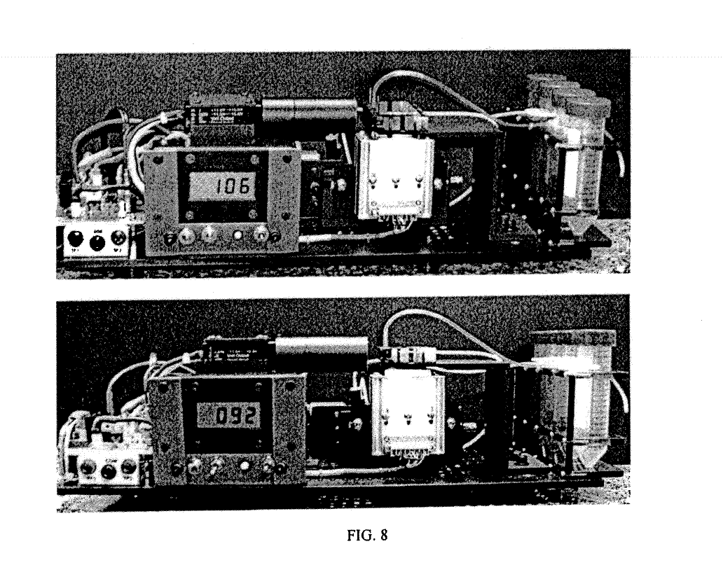

FIG. 8 illustrates two exemplary Figures showing the Cyan and Red CLED in operation.

In another example, a portable incubator using the portable biosensing system is described. As illustrated in FIG. 9, the main components of the portable CO₂ incubator consists of miniature disposable 5% CO₂ premix gas cylinder with regulator, a CO₂ concentration monitor with remote probe, LCD readout, a dual alarm temperature control sensor with two output relay control circuits, a seamless stainless steel enclosure, and rechargeable battery supply. The CO₂ concentration monitor is a microprocessor based, non-dispersive infrared high moisture area, CO₂ concentration sensor which has a 0.1% CO₂ LED reading accuracy. The opto-coupled microprocessor controlled temperature sensor was assembled from a professional grade kit, and has an accuracy of ±0.1° C., dual controllable hysteresis, and LED readout is illustrated in FIG. 10. The chamber will be electrically heated by aluminum block micro heaters to maintain the chamber temperature between 37.5 to 40.0° C. A small positive pressure fan motor is mounted outside of the culturing area to help to circulate the air inside the chamber without disturbing cultures. A moisture retaining material is placed inside the chamber to produce the relative desired humidity levels between 95-98%.

The system of the present invention can produce an array of homogeneously sized cells for in-vitro cell culture. The capillary platform is scalable both in the size of the capillary volume and the number of the capillary 102.

The capillary 102 is a very inexpensive material that can be customized for different applications. The inside of the capillary can be modified with different types of polymeric coatings in order to make it cell-culture friendly, biocompatible, highly permeable to oxygen, as well as allowing optimal environment, sensing chemistry and fluidics handling for mammalian cell culture.

A hybrid of glass, Teflon or hydrogels provides a highly transparent, low fluorescent and disposable culture chamber.

Examples of hydrogels that could be integrated with the glass capillary include polylactic acid, polyacrylic acid (super adsorbents in diapers), polyacrylamide materials (used in contact lens), 2-hydroxypropyl-methacrylate polymer (HPMA-used in drug delivery system).

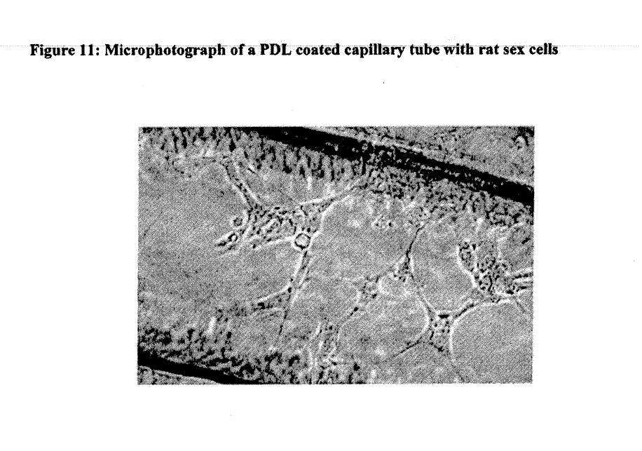

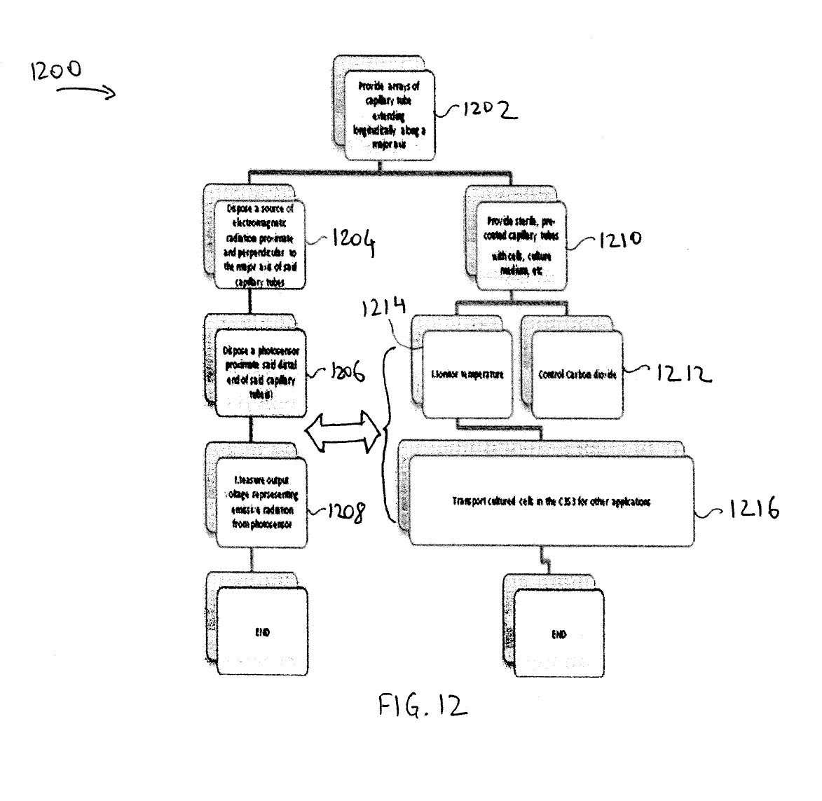

Positively charged polymers that can be used include Chitosan (pKa˜6.5) and polylysine. It is estimated that 6-10 (˜4 cm length) glass capillary chamber can accommodate equivalent wells spaced at 300 μm apart and can currently be fabricated at a materials cost of $0.90. The capillary 102 is optically clear, non-toxic and biocompatible, enabling important gases (Oxygen, Carbon-dioxide) to exchange with the surrounding to support cell-growth. This capillary 102 provides a means of imaging cells directly in bright field or in fluorescence mode as illustrated in FIG. 11.

In addition, there are unique, less obvious advantages of the system of the present invention 100's architecture that are specific to the proposed cell culture applications—capillary 102 has a 3-D geometry, which is sufficient for cell proliferation under controlled conditions. With cylindrical geometries, cells tend to spread at edges as is commonly observed in 2-D tissue culture plate. The narrower opening (vs. conventional cell culture plate) helps to delay the soluble factors produced by the cells in the capillary from diffusing out of the well. This allows the cells to condition quickly to their microenvironment. The whole system incorporates a transfer line (with passive dilution network) into the glass capillary control module capable of generating highly reproducible linear or logarithmic dilutions over a wide range of concentrations. Passive dilutions networks are precise, robust and simple to operate, making them ideal for eventual field use. Moreover, the system of the present invention 100 could be extended to perform viability assays for a wide range of both adherent and non-adherent cells. In summary, the system of the present invention 100 provides significant advantages over conventional cell culture platform. These include low cost, good optical properties, ease of sterilization and the small amount of reagents required.

System 100 creates a novel tool for researchers involved in cell to cell interactions, cell-based biosensors, cell-substrate interaction, drug testing, cytotoxicity, cytocompatibility, regenerative medicine, tissue culture and engineering. Several examples of these applications are discussed below:

1. Cell-based Biosensors: Food borne pathogens pose a risk to food safety and are a threat to the global food supply chain. Correct detection and identification of food borne pathogens and other contaminants relying on conventional culturing techniques are very elaborate, time-consuming, and have to be completed in a microbiology laboratory. Automation in detection methods of food pathogens is highly desirable. Therefore, biosensor-based tools offer the most promising solutions and address some of the modern-day needs for fast and sensitive detection of pathogens in real time or near real time. The need for a more rapid, reliable, specific, and sensitive method of detecting a target analyte, at low cost, is the focus of a great deal of research. In particular, system 100 provides a means to culture food-borne pathogens under stringent environmental controls.

2. Optical Imaging and Spectroscopic Techniques: These techniques have recently attracted a lot of interest for medical applications due to its non-invasive procedure, high temporal resolution and relative low cost. Fluorescence imaging in the visible-wavelength range is routinely used for conventional cell microscopy because cellular uptake is a necessary prerequisite. One simple cytotoxicity test involves visual inspection of the cells with bright field microscopy for changes in cellular or nuclear morphology. System 100 can be used for fluorescence spectroscopy using different types of reagents such as calcein AM and Alamar Blue. These are important fluorescence dyes (calcein acetoxymethyl (calcein AM) and ethidium homodimer) commonly used to test the live/dead viability test when exposed to drugs and toxins. When excited at 495 nm, calcein AM and ethidium homodimer emit distinct fluorescence signatures at 515 nm and 635 nm, respectively. These wavelengths are available in the system 100 for such applications. Alamar Blue is reduced to a soluble fluorescent product, resorufin (λₑₘ 590 nm), providing simpler sample preparation compared to the MTT assay. However, interpretation of Alamar Blue results may be difficult because the biochemical mechanisms of Alamar Blue reduction have not yet been explored. In the case of cytotoxicity, it is to be noted that cell cultures are sensitive to changes in their environment such as fluctuations in temperature, pH, and nutrient, in addition to the concentration of the potentially toxic agent being tested. Therefore, controlling the experimental conditions is crucial so as to ensure that the measured cell death corresponds to the toxicity of the added analytes versus the unstable culturing conditions.

3. Classical Cytotoxicity Assays: In vitro cytotoxicity assays are the major alternatives to animal testing for basal cytotoxicity assessment of chemicals, typically indicating the number of cells which are dead or alive after exposure to test chemicals. Conventional in vitro cell-based assays are commonly used to screen cytotoxic effects induced by chemicals in a variety of cell systems. Examples include biochemical methods such as (3-(4,5-dimethylthiazol-2-yl)-2,5-diphenyltetrazolium bromide test, MTT), neutral red uptake (NRU), ATP and lactate dehydrogenase (LDH) measurement, Sulforhodamine B (SRB) assay, WST assay. Others include growth assays such as colony forming efficiency (CFE), cytokine assay, phagocytosis assay, nitric oxide assay, and glutathione assays. The present invention is equally applicable to measure the cytoxicity of drugs, toxins, bioweapons and nanoparticles.

4. Cell Viability Testing: Exposure to certain cytotoxic agents can compromise the cell membrane, which allows cellular contents to leak out. Viability tests based include neutral red, toluoylene red, and many more. Toluene is a weak cationic dye that can cross the plasma membrane by diffusion. The dye tends to accumulate in lysosomes within the cell. If the cell membrane is altered, the uptake of the dye decreased and can leak out, allowing discernment between live and dead cells. Cytotoxicity can be quantified by taking spectro-photonic measurements of the neutral red uptake under varying exposure conditions. 2-(4,5-dimethyl-2-thiazolyl)-3,5-diphenyl-2H-tetrazolium bromide (MTT) assay is among the most versatile and popular assays used for in vitro toxicology. Mitochondrial activity can be tested using tetrazolium salts as mitochondrial dehydrogenase enzymes cleave the tetrazolium ring and this reaction only occur in living cells. Reduction of water-soluble MTT salt by metabolically active cells leads to the formation of MTT-formazan crystals. The insoluble MTT-formazan is deposited in mitochondria, in the cytoplasm, and in the regions of plasma membranes. Reduction of MTT in isolated cells is regarded as an indicator of “cell redox activity”. This technique has many advantages when compared to other toxicity assays, because it requires minimal physical manipulation of the model cells and yields quick, reproducible results requiring simple optical density acquisition. Other tetrazolium-based assays used to test the cytotoxicity are the MTS, XTT or WST assay. The number of living cells can be determined similarly by quantifying the production of soluble formazan. In assays that produce insoluble formazan dyes (such as the MTT assay), exocytosis of the crystalline product can skew results; therefore assays that produce soluble dyes (such as MTS, XTT or WST-1) are preferred. System 100 provides template for clean cell growth and quantitation.

5. Cell-based microfluidic devices: System 100 is a unique instrument integrating microfluidics with cell culture-based assays, and can be used as cell microreactors.

6. Cell differentiation: System 100 is an ideal template for cell monitoring cell differentiation (germ cells, somatic cell or stem cells) and de-differentiation: Myocites, osteoblast, adipocytes, neurocytes). System 100 can be used for examining cytocompatibility (improved implant component maintenance of cell functions) and regenerative medicine. In regenerative medicine, tissues or organ functions that have been lost due to age, disease, damage or other defects can be regenerated by stimulating the dead organs to heal themselves. System 100 can empower one of ordinary skill in the art after reading this disclosure to grow tissues or organs in the laboratory, allow safe transportation of life-saving organs and ultimately safely implant them when the body cannot heal itself.

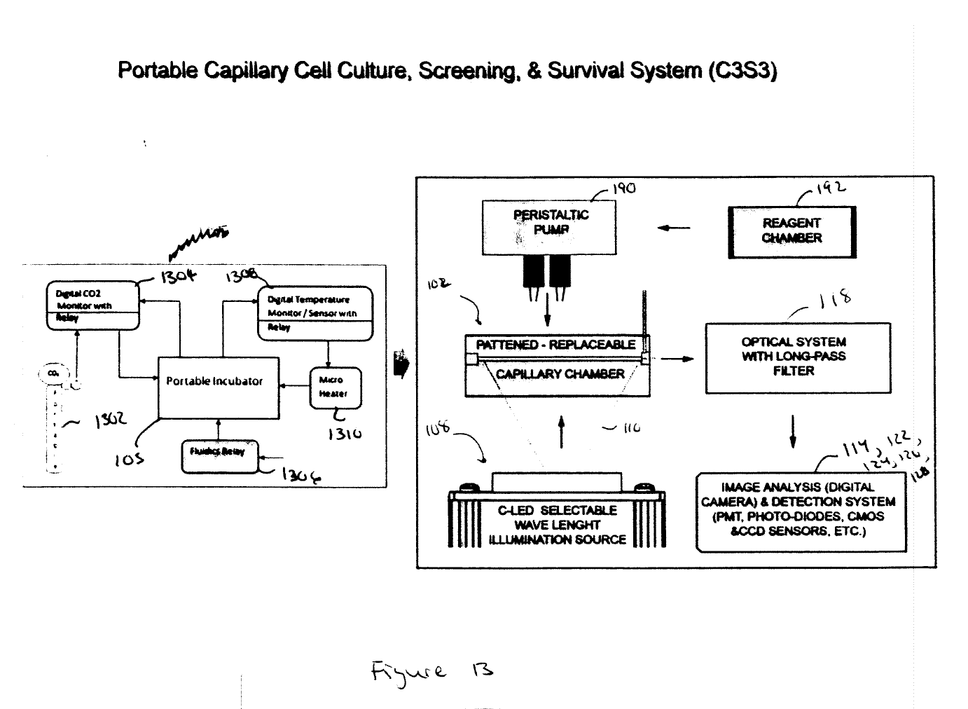

Referring to FIG. 12, a flowchart 1200 for a portable biosensing method according to various embodiments of the present invention is disclosed. The flowchart 1200 begins at step 1202 where a capillary tube 102 (or, capillary tube array 188) extending longitudinally along a major axis between a proximal inlet end and a distal end is provided. As discussed above with reference to FIGS. 1A-11, capillary 102 has an interior surface coated with a capture material and forms a waveguide. In step 1204, a CLED 108 proximate and perpendicular to the major axis of capillary tube 102 is disposed and positioned relative to the capillary tube 102 so that energy (e.g., excitation 110) enters capillary tube 102 from its exterior along the entire length of capillary tube 102 to project a line of energy along substantially the entire longitudinal extent of capillary tube 102. In step 1206, a photosensor (e.g., PMT 114) is disposed proximate said distal end of capillary tube 102 for receiving emissive radiation therefrom, photosensor generating an output voltage representative of the emissive radiation. In step 1208, output voltage indicative of emissive radiation from the capillary tube 102 is measured.

Flowchart 1200 further illustrates a method including steps 1210-1216 used in parallel with steps 1202-1208 described above. In step 1210, sterile, pre-coated capillary tube(s) with cell cultures, culture mediums and other biological and chemical materials is provided. In step 1214, temperature of C3S3 system 103 of FIGS. 1A and 1E is monitored using temperature controller 196 in parallel with step 1212 where carbon dioxide content of the portable incubator 250 is controlled. The flow then proceeds to step 1216 where cultured cells in C3S3 system 103 are transported for other applications, for example, preservation, or further treatment. Further, in one embodiment, the portable carbon dioxide incubator 250 is configured to work with system 100 as illustrated in FIG. 1E.

FIG. 13 is a schematic drawing of the system of the present invention showing further details of the incubator components. Amongst the components are digital CO₂ monitor 1304 with CO₂ cylinder 1302, temperature sensor 1308 with heater 1310, and fluidic relay 1306.

CO₂ monitor 1304 is needed, because CO₂ gas plays an important role in regulating the pH through a bicarbonate buffer system. Normally, CO₂ monitor 1304 maintains a 5-7% level of CO₂. Higher or lower levels will indirectly induce a change in the cell culture medium pH value, so a constant CO₂ concentration is maintained by CO₂ monitor 1304. The CO₂ gas is stored in CO₂ cylinder 1302.

The optimal incubation temperature varies according to cell type, but most mammalian cell lines will grow satisfactorily at 37° C. which is similar to human body temperature. Thus, temperature sensor 1308 regulates the operating temperature at a level of 37° C. within ±0.5° C. In addition, the power of CLED beam used for the optical detection should also be chosen in a manner that avoids overheating the cells. Different cell lines use sera with different chemical components. Care must be taken while injecting solution into capillary containing cells. Air bubbles may induce cell death due to the high osmotic stresses across the cell membrane. Fluidic relay 1306 controls the flow of these materials into the capillary system.

Also shown in FIG. 13 is peristaltic pump 190, which controls the flow of liquid, and reagent chamber 192 which stores liquids being pumped into the system. All other optical components in FIG. 13 have been described above with the capillary chamber being the storage rack for patterned or coated capillaries.

### EXAMPLES

### Example 1

### Cell Culture, Growth and Survival Application for Mammalian Cell Culture

The application of the device of the present invention to sustain the culture of IEC-6, A549 and TM-4 has been investigated. It has been found that these cell lines can be successfully cultured inside a 0.1% PDL (Poly-D-lysine) or 1 mg PDL/10 mL sterile water coated capillary (ID=1.0 mm, OD=0.78 mm, length=38 mm). The optimum number of each cell lines in the capillary varies (typically around 7000 cells/capillary). It has also been found that A549 can survive within the capillary for at least 14 days. These results had been verified using Trypan Blue assay. It was observed that different concentrations of PDL have no direct effect on how well the cells can survive inside the capillary 102. The cell-lines used here are the adhesion type, which require attachment to a substrate in order to seed, survive, spread, and grow. Cells typically do not attach to substrates whose surfaces are uncharged or hydrophobic. Hence, the traditional cell culture substrates are specially pre-treated, single use, disposable plastics such as polystyrene. Those substrates all have planar surfaces. The system of the present invention is by no means limited to adherent cell cultures, non-adherent cells can also be used including bacteria, fungi, viruses, and nucleic acids. It has been demonstrated that cells can be maintained and proliferated under controlled conditions in the glass capillary. Different surface treatments were investigated to allow media exchange, cell adhesion and motility while keeping the cells in place: poly-L-lysine, concanavalin A, and polyamic acid. The coating procedure used is outlined below.

### Example 2

### Polymer Coating Procedures

The interior of the capillary tube 102 of the present invention is coated in the following exemplary fashion:

1. Borosilicate capillary glass tubing (ID=0.78 mm, OD=1.00 mm, Length=39 mm) was autoclaved at 134° C. for 35 minutes.

2. 1 mg Poly-D-lysine hydrobromide purchased from Sigma-Aldrich was dissolved in 10 mL sterile tissue culture grade water.

3. The dissolved solution was injected into sterile capillary glass tubing by using BD Ultra-Fine Insulin Syringe (capacity: 0.5 ml, length: 12.7 mm, gauge: 30 G). The capillary was rocked gently to ensure even coating inside the capillary.

4. Capillary 102 was thoroughly rinsed after 5 minutes using sterile tissue culture grade water. The liquid residues were removed by aspiration.

5. Capillary 102 was allowed to dry overnight inside a Laminar Flow hood (Ductless PCR workstation) before introducing cells and medium.

### Example 3

### Analysis of Cells

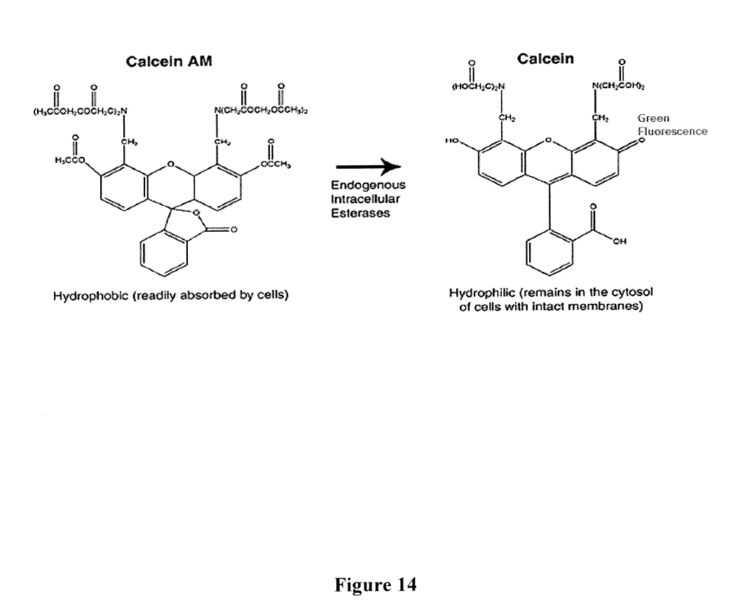

Capillaries are coated by adding 10 mL of sterile tissue culture grade water to 1 mg of poly-D-Lysine at pH 7.32. The resulting solution is introduced to sterile glass capillaries via ultrafine insulin syringe. The capillaries are rocked gently to promote even coating and drying. Then, the inner surfaces of the capillaries are thoroughly rinsed with sterile tissue culture grade water to remove any residue. The coated capillaries are dried overnight before introducing cells. Following successful polymer coating, the capillaries are seeded with mammalian cells to allow growth. Typically, a few cells (5-10) per capillary is equivalent to a plating density of 5000-7,000 cells per standard 96 well (0.32 cm²) tissue culture plate. Capillaries are kept within the C3S3 compartment under controlled conditions (37° C., 5% CO₂) and the culture media are changed periodically until the cells reach confluence. Cell viability is then determined using any cell-permeable compound such as Calcein-AM (Sigma-Aldrich Inc. USA) after transferring the cell-coated capillaries into the detection compartment of the C3S3 system of the present invention. As shown in FIG. 14, this Calcein AM only enters viable cells (dead cells are not affected) and is converted by intracellular esterases to calcein which can be monitored at Ex/Em=490 nm/510 nm. Because this photostable calcein is well retained in viable cells and could be influenced by the intracellular pH fluctuation, it has been widely used for quantifying cell numbers and for studying cell migration and other cell-based assays.

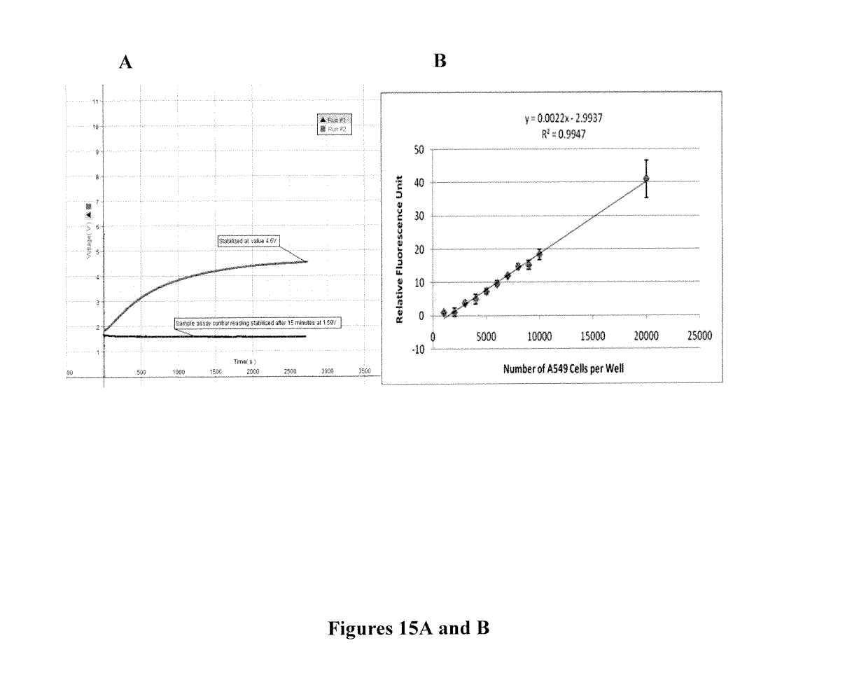

Cell counting is accomplished in the C3S3 system of the present invention using 2.2 μM Calcein AM at 40 minutes optimum incubation time while comparison is carried out using conventional Trypan Blue Exclusive Assay. FIG. 15 shows the typical calibration curve recorded at 12 different cell densities: 1000, 2000, 3000, 4000, 5000, 6000, 7000, 8000, 9000, 10000, and 20000 cells/well, respectively. Each density was measured 6 times using DPBS (buffered cell medium) alone and with 2.2 μM Calcein AM as the sample assay control. Comparative analysis was carried out using a standard microplate while fluorescence readings were taken at 5, 10, 20, 40, and 60 minute intervals. The basic operation steps are:
Place capillary containing mammalian (e.g. A549) cells into the CLED-UPAC system (see FIG. 1D)
Rinse capillary by flowing in the DPBS (buffer) using a pump
Turn on the CLED for 40 minutes to generate a baseline (sample assay control) data
Turn off the CLED and flow freshly prepared 2.2 μM Calcein AM into the capillary
Turn on the CLED to run a kinetic study for an additional 40 minutes.

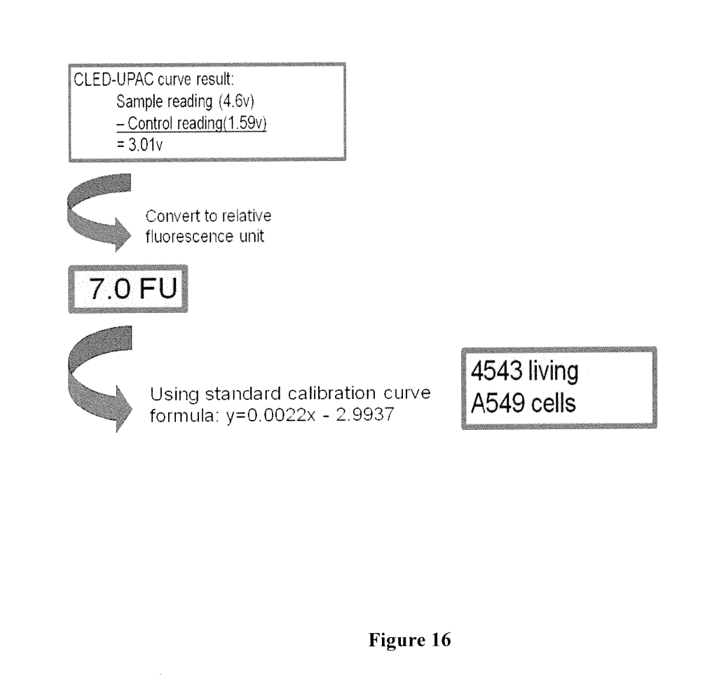

The generated data was treated by subtracting sample reading from assay control reading which as shown in FIG. 16. The obtained value is converted to the relative fluorescence unit of the microplate reader by using optical parameters provided by the manufacturer. The standard calibration curve showed that there were 4543 living A549 cells inside this capillary. Results showed that comparable cell viability was detected using the C3S3 and Tryphan Blue assays. This shows that CLED-UPAC system provides the possibility of cell viability testing.

Since other modifications and changes varied to fit particular operating requirements and environments will be apparent to those skilled in the art, the present invention is not considered limited to the example chosen for purposes of disclosure, and covers all changes and modifications which do not constitute departures from the true spirit and scope of this invention.

Having thus described the basic concept of the present invention, it will be apparent to those of ordinary skill in the art that the foregoing detailed disclosure is intended to be presented by way of example only, and is not limiting. Various alterations, improvements, and modifications will occur and are intended to those of ordinary skill in the art, though not expressly stated herein. These alterations, improvements, and modifications are intended to be suggested hereby, and are within the spirit and scope of the present invention. Two or more components of the system 100 can be integrated, or may be made parts of an integrated circuit chip. Further, alterations in electrical and mechanical components may be realized by interchanging and/or adding electrical connections and components for mechanical connections or components and vice-versa, as and when appropriate without departing from the scope of various exemplary aspects of the present invention, as described above. Additionally, the recited order of processing elements or sequences, or the use of numbers, letters, or other designations therefore, is not intended to limit the claimed processes to any particular order.

What is claimed:

1. A portable biosensor system, comprising:
a) at least one capillary tube extending longitudinally along a major axis between a proximal inlet end and a distal end, said at least one capillary tube having an interior surface coated with a capture material and forming a waveguide;
b) at least one collimated light emitting diode disposed proximate and perpendicular to the major axis of said at least one capillary tube and positioned relative to the at least one capillary tube so that energy enters said at least one capillary tube from its exterior along the entire length of said at least one capillary tube to project a line of energy along substantially the entire longitudinal extent of said at least one capillary tube;
c) a photosensor disposed proximate said distal end of said at least one capillary tube for receiving emissive radiation therefrom, said photosensor generating an output voltage representative of said emissive radiation; and
d) means for measuring said output voltage.

2. The portable biosensor system of claim 1, wherein said at least one capillary tube comprises means for introducing a fluid into said at least one capillary tube, means for extracting a fluid from said capillary tube, or combinations thereof.

3. The portable biosensor system of claim 1, wherein said capture material comprises a capture antigen.

4. The portable biosensor system of claim 3, wherein said capture antigen comprises at least one material selected from the group consisting of DNA, RNA, whole cells, carbohydrates, and lectins.

5. The portable biosensor system of claim 1, wherein said means for measuring comprises means for displaying a value representative of said output voltage.

6. The portable biosensor system of claim 5, wherein said means for measuring said output voltage comprises an integrating voltmeter.

7. The portable biosensor system of claim 6, wherein said integrating voltmeter comprises an A/D converter.

8. The portable biosensor system of claim 5, wherein said means for displaying a value comprises a digital display.

9. The portable biosensor system of claim 1 further comprising:
e) an optical arrangement disposed intermediate said distal end of said at least one capillary tube and said photosensor.

10. The portable biosensor system of claim 9, wherein said optical arrangement comprises at least one lens, an optical filter, and combinations thereof.

11. The portable biosensor system of claim 9, wherein said at least one capillary tube, said optical arrangement, and said photosensor are substantially axially aligned.

12. The portable biosensor system of claim 10, wherein said at least one lens comprises a plano-convex lens.

13. The portable biosensor system of claim 10, wherein said optical filter comprises a low-pass optical filter.

14. The portable biosensor system of claim 1, wherein said photosensor comprises a photodetector assembly, said photodetector assembly comprises a photomultiplier tube, a photodiode, a optical fiber coupled photo spectrometer, a digital imaging device including at least one of a complementary metal oxide semiconductor (CMOS) device, a charge coupled device (CCD), and a digital imager, and at least one non-visible wavelength detector including at least one of an infrared detector and an ultraviolet detector.

15. The portable biosensor system of claim 6 further comprising:
e) a low-pass electrical filter disposed intermediate said photosensor and said integrating voltmeter.

16. The portable biosensor system of claim 15, wherein said low-pass filter comprises a Butterworth filter.

17. The portable biosensor system of claim 1 further comprising:
e) a computer interface adapted to present a signal representative of said output voltage to a device external to said portable biosensor.

18. The portable biosensor system of claim 2 further comprising:
e) means for pumping operatively connected to at least one of said means for introducing a fluid into said capillary tube, and said means for extracting a fluid from said capillary tube.

19. The portable biosensor system of claim 18, wherein said means for pumping comprises a multi-speed, peristaltic pump.

20. The portable biosensor system of claim 9 further comprising:
f) a vibration-isolating mounting structure for supporting at least one of: said capillary tube, said at least one collimated light emitting diode, and said photosensor, and said optical arrangement.

21. The portable biosensor system of claim 1, wherein the collimated light emitting diode comprises at least one of a collimating lens coupled to the at least one light emitting diode, a short pass filter coupled to the collimating lens, or combinations thereof.

22. The portable biosensor system of claim 1, wherein the at least one light emitting diode comprises at least two wavelength selectable light emitting diodes each with respective driver modules in a housing.

23. The portable biosensor system of claim 22, wherein the at least two light emitting diodes are mounted on a heat sink and are in a relative angular orientation with respect to each other.

24. The portable biosensor system of claim 22, wherein the housing comprises a cylindrical lens on an outside surface arranged to receive electromagnetic radiation from the at least two wavelength selectable light emitting diodes.

25. The portable biosensor system of claim 22, wherein at least one of the driver modules is a dc to dc converter controllable by an external voltage control device.

26. The portable biosensor system of claim 1, wherein the at least one capillary tube comprises a plurality of capillary tubes arranged in a surface designed glass capillary array.

27. The portable biosensor system of claim 1, further comprising:
an incubator for incubation of cells in said capillary tube.

28. A method for detecting presence of a target molecule in a sample, said method comprising:
providing a biosensor system comprising:
at least one capillary tube extending longitudinally along a major axis between a proximal inlet end and a distal end, said at least one capillary tube having an interior surface coated with a capture material and forming a waveguide;
at least one collimated light emitting diode proximate and perpendicular to the major axis of said at least one capillary tube and positioned relative to the at least one capillary tube so that energy enters said at least one capillary tube from its exterior along the entire length of said at least one capillary tube to project a line of energy along substantially the entire longitudinal extent of said at least one capillary tube; and
a photosensor proximate said distal end of said at least one capillary tube for receiving emissive radiation therefrom, said photosensor generating an output voltage representative of said emissive radiation;
passing the sample through the at least one capillary tube;
directing electromagnetic radiation emitted from the at least one collimated light emitting diode to the at least one capillary tube;
receiving radiation emitted from the at least one capillary tube with the photosensor;
detecting any target molecule present in the sample based on the radiation received by the photosensor.

29. The method of claim 28, wherein the capture material comprises a capture antigen.

30. The method of claim 29, wherein the capture antigen comprises at least one material selected from the group consisting of DNA, RNA, whole cells, carbohydrates, and lectins.

31. The method of claim 28, wherein said detecting is carried out based on the output voltage generated by the photosensor.

32. The method of claim 28, wherein the biosensor system comprises:
an optical arrangement intermediate said distal end of said at least one capillary tube and said photosensor.

33. The method of claim 28, wherein the biosensor system comprises:
a computer interface adapted to present a signal representative of said output voltage to a device external to said portable biosensor.

34. The method of claim 28, wherein said passing the sample through the at least one capillary tube comprises:
pumping the fluid into said at least one capillary tube.

35. The method of claim 34, wherein said pumping is carried out using a multi-speed, peristaltic pump.

36. The method of claim 28, wherein the collimated light emitting diode comprises at least one of a collimating lens coupled to the at least one light emitting diode, a short pass filter coupled to the collimating lens, or combinations thereof.

37. The method of claim 28, wherein the at least one light emitting diode comprises at least two wavelength selectable light emitting diodes each with respective driver modules in a housing.

38. The method of claim 28, wherein the at least one capillary tube comprises a plurality of capillary tubes arranged in a surface designed glass capillary array.

39. The method of claim 28, wherein the biosensor system further comprises:
an incubator for incubation of cells in said at least one capillary tube.
